(19) United States
(12) Patent Application Publication
(10) Pub. No.: US 2021/0361598 A1
(43) Pub. Date: Nov. 25, 2021

(54) COMPOUNDS FOR PREVENTION AND TREATMENT OF POST-INTENSIVE CARE COGNITIVE DYSFUNCTION AND COGNITIVE DYSFUNCTION RESULTING FROM RESPIRATORY DISTRESS

(71) Applicant: Abrexa Pharmaceuticals, Inc., San Deigo, CA (US)

(72) Inventors: Jesus Ernesto VILLAFRANCA, San Diego, CA (US); Charles KISSINGER, San Diego, CA (US)

(21) Appl. No.: 17/242,755

(22) Filed: Apr. 28, 2021

Related U.S. Application Data
(60) Provisional application No. 63/016,724, filed on Apr. 28, 2020.

(51) Int. Cl. A61K 31/167; A61P 25/28

(52) U.S. Cl. CPC A61K 31/167; A61P 25/28

(57) ABSTRACT

Presented herein, in certain aspects, are methods of preventing, reducing, delaying and treating Post-Intensive Care Cognitive Dysfunction (PICCD) by administering a compound disclosed herein to a subject prior to, during and/or after a stay in an ICU, intubation or connection to a ventilator. Also presented herein, in certain aspects, are methods of treating, preventing, inhibiting, reducing the severity of, or delaying the onset of a cognitive impairment or a cognitive disorder resulting from, or caused by respiratory distress.

### RELATED PATENT APPLICATIONS

This application claims priority to U.S. Provisional Patent Application No. 63/016,724, filed Apr. 28, 2020, the contents of which are incorporated herein by reference in its entirety.

### FIELD OF THE INVENTION

Provided herein, in certain aspects, are methods for the treatment and/or prevention of Post-Intensive Care Cognitive Dysfunction (PICCD) that comprise administering a compound disclosed herein to a subject prior to, during and/or after a stay in an ICU, intubation or connection to a ventilator. Also provided herein, in certain aspects, are methods of treating or preventing cognitive dysfunction resulting from, or caused by respiratory distress, which methods comprise administering a compound disclosed herein to a subject prior to, during and/or after respiratory distress.

### BACKGROUND

Post-Intensive Care Cognitive Dysfunction (PICCD) is an impairment or decline in cognitive function observed in a subject after a stay in an intensive care unit (ICU), after intubation and/or after connection to a ventilator. Patients who experience respiratory distress, such as acute respiratory distress syndrome (ARDS), also experience an impairment or decline in cognitive function during or after respiratory distress. Compounds disclosed herein can be used treat and/or prevent cognitive dysfunction associated with PICCD and respiratory distress.

### SUMMARY

In certain aspects, provided herein is a method of preventing, reducing, delaying or treating Post-Intensive Care Cognitive Dysfunction (PICCD) in a subject comprising administering to the subject a therapeutically effective amount of a compound disclosed herein. In certain embodiments, the PICCD comprises a presentation of, or worsening of: delirium, memory loss, a confusional state, reduced awareness, impaired executive function, impaired speech, impaired language, impaired communication, loss of attention, depression, anxiety, post-traumatic stress disorder and/or an impairment of visual-spatial abilities. In certain embodiments, the PICCD is a result of, is associated with, or is induced by: a) a subject's stay in or admittance to an intensive care unit, b) intubation of the subject, and/or c) operably connecting the subject to a ventilator.

In certain aspects, provided herein is a method of treating, preventing, inhibiting, reducing the severity of, or delaying the onset of a cognitive impairment or a cognitive disorder resulting from, or caused by respiratory distress in a subject, the method comprising administering a therapeutically effective amount of a compound disclosed herein to a subject who is at risk of having respiratory distress, who has or is experiencing respiratory distress, or who has previously experienced respiratory distress.

In certain aspects, provided herein is a method of preventing or inhibiting a decline of, or a worsening of, a pre-existing cognitive impairment or a cognitive disorder resulting from, or caused by respiratory distress in a subject, the method comprising administering a therapeutically effective amount of a compound disclosed herein to a subject who is at risk of having respiratory distress, who has or is experiencing respiratory distress, or who has previously experienced respiratory distress.

In certain aspects, provided herein is a method of treating a subject who is at risk of having respiratory distress, who has or is experiencing respiratory distress, or who has previously experienced respiratory distress comprising administering a therapeutically effective amount of a compound disclosed herein to the subject, wherein a cognitive impairment or a cognitive disorder resulting from, or caused the respiratory distress is prevented, ameliorated, inhibited, reduced in severity, or delayed.

In certain aspects, provided herein is a method of treating a subject who is at risk of having respiratory distress, who has or is experiencing respiratory distress, or who has previously experienced respiratory distress comprising administering a therapeutically effective amount of a compound disclosed herein to the subject, wherein a pre-existing cognitive impairment, a pre-existing cognitive disorder, or a pre-existing neurodegenerative disease is preventing from worsening, or preventing or inhibited from increasing in severity.

In some embodiments, the subject has, is suspected of having, is diagnosed with, or is at risk of having acute respiratory syndrome, acute respiratory distress syndrome (ARDS), severe acute respiratory syndrome, asthma, pneumonia, or an infection.

In some embodiments, the subject is infected with a coronavirus. In some embodiments, the subject has or is suspected of having COVID-19.

In some embodiments, a compound disclosed herein is a compound of Formula I, II, III or IV. In some embodiments, a compound comprises the structure of Formula IV;

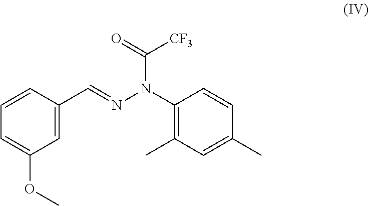 [SMILES: COc1cccc(/C=N/N(C(=O)C(F)(F)F)c2ccc(C)cc2C)c1]

or a pharmaceutically acceptable salt, stereoisomer or tautomer thereof.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments. The patent or application file contains at least one drawing executed in color.

Copies of this patent or patent application publication with color drawing(s) will be provided by the Office upon request and payment of the necessary fee.

FIG. 1 illustrates the experimental design of Example 3, with mice subjected to ischemia and 45 minutes of hypoxia.

FIG. 2 illustrates in Example 3 the 2,3,5-triphenyltetrazolium chloride (TTC) staining of the mouse hippocampus after 24 hour of HI injury in representative brain coronal sections where white indicates damage areas (left) and MRI imaging measurements of infarction volume as a percentage of the contralateral hemisphere volume for SH (sham control mice), HI (hypoxic-ischemic mice untreated) and HIJ (hypoxic-ischemic mice treated with J147). The figure shows that J147 significantly reduces the area of brain injury due to hypoxia-ischemia.

FIG. 3 illustrates in Example 3 the effect of treatment with J147 through the magnetic resonance imaging measurement of ipsilateral hemisphere volume as a percentage of contralateral hemisphere volume with the degree of reduction from 100% reflecting the magnitude of the infarct volume 50 days after hypoxia-ischemia injury. The figure shows that 50 days after injury J147 almost entirely reduces the area of brain injury due to hypoxia-ischemia.

FIG. 4 illustrates in Example 3 the analysis of the dose dependency of J147 treatment for the reduction of apoptotic neuronal cell death in hippocampus 4 days after injury in a neonatal hypoxic-ischemic encephalopathy mouse model.

FIG. 5 illustrates in Example 3 the reduction 4 days after injury in apoptotic cell death from J147 treatment through measurement of NeuN+ area and counting TUNEL+ cells in hippocampus in sham-treated mice, HI mice and HI mice treated with J147.

FIG. 6 illustrates in Example 3 the functional recovery of HI mice induced by treatment with J147 in open field, rotarod, and y-maze testing.

### DETAILED DESCRIPTION

Presented herein, in some embodiments, are compounds for treating and/or preventing PICCD. Post-Intensive Care Cognitive Dysfunction (PICCD) is an impairment or decline in cognitive function observed in a subject after a stay in an intensive care unit (ICU), after intubation and/or after connection to a ventilator. Presented herein, in some embodiments, are compounds for treating and/or preventing cognitive impairment or a decline in cognitive function observed in a subject during or after respiratory distress.

### Compounds

In some embodiments provided herein is a compound for use in preventing or treating PICCD. In some embodiments, a compound for use herein comprises the structure of Formula I;

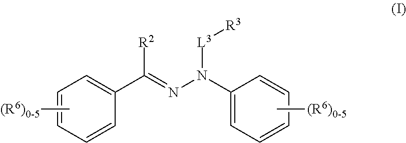

or a pharmaceutically acceptable salt, stereoisomer or tautomer thereof. In some embodiments of Formula I, R² is hydrogen (H) or methyl; R³ is a methyl, a fluorine substituted alkyl (e.g., fluoromethyl, difluoromethyl, or trifluoromethyl), or a bromine substituted alkyl (e.g., bromomethyl, dibromomethyl, tribromomethyl); L³ is a carbonyl; and R⁶ at each occurrence is independently selected from alkyl, substituted alkyl, alkenyl, substituted alkenyl, cycloalkyl, substituted cycloalkyl, hydroxyl, methoxy, alkoxy, substituted alkoxy, aryloxy, substituted aryloxy, mercapto, alkylthio, arylthio, carbonyl, carboxyl, aryl, substituted aryl, substituted heterocyclic, halogen, cyano, cyanoalkyl, amine, methyl amine, dimethyl amine, nitro, amino, amidino, carbamate, CF₃, OCF₃, S(O)ₙR⁷, and C(O)R⁸, or two R⁶ at adjacent positions combine to form an optionally substituted heteroaryl or heteroalkyl ring fused with the adjoining phenyl moiety; where R⁷ is selected from H, R⁹, NH₂, HNR⁹ and NR⁹R¹⁰; R⁸ is selected from OH, OR⁹, NH₂, NHR⁹ and NR⁹R¹⁰; where R⁹ and R¹⁰ at each occurrence are independently an optionally substituted alkyl; and n is 1 or 2.

In certain embodiments of Formula I, R⁶ at each occurrence is independently selected from alkyl, substituted alkyl, alkenyl, substituted alkenyl, hydroxyl, alkoxy, methoxy, substituted alkoxy, halogen, carbonyl, carboxyl, or C(O)R⁸; and in certain such aspects, R⁶ at each occurrence is methyl, methoxy, perfluoromethyl, perfluoromethoxy, hydroxyl, Cl, F, or I. In some embodiments of Formula I, L³ is carbonyl, R³ is CF³, R² is H, and R⁶ is null or H at every occurrence. In some embodiments of Formula I, L³ is carbonyl, R³ is CF₃, R² is H, and R⁶ is independently selected from methyl or methoxy, at each occurrence. In some embodiments of Formula I, L³ is carbonyl, R³ is CF₃, R² is methyl, and R⁶ is independently selected from methyl or methoxy, at each occurrence.

In some embodiments, a compound for use herein comprises the structure of Formula II;

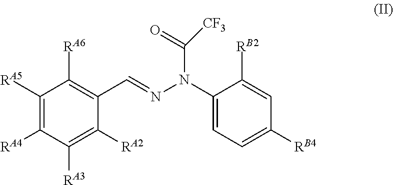

or a pharmaceutically acceptable salt, stereoisomer or tautomer thereof, where:

(i) R^{A2}, R^{A4}, R^{A5}, and R^{A6} is H, R^{A3} is methoxy, R^{B2} is methyl, and R^{B4} is methyl;

(ii) R^{A2}, R^{A3}, R^{A5}, and R^{A6} is H, R^{A4} is methoxy, R^{B2} is methyl, and R^{B4} is methyl;

(iii) R^{A2}, R^{A3}, R^{A4}, R^{A5}, and R^{A6} is H, R^{B2} is H, and R^{B4} is H;

(iv) R^{A2}, R^{A3}, R^{A4}, R^{A5}, and R^{A6} is H, R^{B2} is methyl, and R^{B4} is methyl;

(v) R^{A2}, R^{A4}, R^{A5}, and R^{A6} is H, R^{A3} is methoxy, R^{B2} is H, and R^{B4} is H;

(vi) R^{A2}, R^{A3}, R^{A4}, R^{A5}, and R^{A6} is H, R^{B2} is H, and R^{B4} is methyl;

(vii) R^{A2}, R^{A4}, R^{A5}, and R^{A6} is H, R^{A3} is methoxy, R^{B2} is H, and R^{B4} is methyl;

(viii) R^{A2}, R^{A3}, R^{A4}, R^{A5}, and R^{A6} is H, R^{B2} is methyl, and R^{B4} is H;

(ix) R^{A2}, R^{A4}, R^{A5}, and R^{A6} is H, R^{A3} is methoxy, R^{B2} is methyl, and R^{B4} is H;

(x) R^{A2}, R^{A3}, R^{A5}, and R^{A6} is H, R^{A4} is COOH, R^{B2} is methyl, and R^{B4} is methyl;

(xi) R^{A2}, R^{A4}, and R^{A5} is H, R^{A3} and R^{A6} is hydroxyl, R^{B2} is methyl, and R^{B4} is methyl;

(xii) R^{A2}, R^{A4}, and R^{A6} is H, R^{A3} and R^{A5} is hydroxyl, R^{B2} is methyl, and R^{B4} is methyl;

(xiii) R^{A2}, R^{A4}, and R^{A5} is H, R^{A3} is methoxy, R^{A6} is F, R^{B2} is H, and R^{B4} is Cl;

(xiv) R^{A3} and R^{A5} is H, R^{A2} and R^{A6} is F, R^{A4} is hydroxyl, R^{A6} is F, R^{B2} is H, and R^{B4} is F;

(xv) R^{A2}, R^{A4}, and R^{A6} is H, R^{A3} is hydroxyl, R^{A5} is F, R^{B2} is H, and R^{B4} is F; or

(xvi) R^{A2}, R^{A5}, and R^{A6} is H, R^{A3} and R^{A4} taken together are —O—CH₂—O—, R^{A5} is F, R^{B2} is H, and R^{B4} is F.

In some embodiments of the compound of Formula II, R^{A2}, R^{A5}, and R^{A6} are H, R^{A3} is methoxy, R^{B2} and R^{B4} are methyl, and R^{A4} is selected from H, NO₂, OH, methoxy, phenol, methyl, Fluorine (F), N(CH₃)₂, CHC(CN)₂ and O-tert-butyldimethylsilyl (OTBDMS). In some embodiments of the compound of Formula II, R^{A2}, R^{A4}, R^{A5}, and R^{A6} are H, R^{A3} is methoxy, R^{B2} is methyl, and R^{B4} is methyl. In some embodiments of the compound of Formula II, R^{A2}, R^{A3}, R^{A5}, and R^{A6} are H, R^{A4} is methoxy, R^{B2} is methyl, and R^{B4} is methyl. In some embodiments of the compound of Formula II, R^{A2}, R^{A3}, R^{A4}, R^{A5} and R^{A6} are H, R^{B2} is methyl, and R^{B4} is methyl. In some embodiments of the compound of Formula II, R^{A2}, R^{A4}, R^{A5} and R^{A6} are H, R^{A3} is methoxy, R^{B2} is H, and R^{B4} is H. In some embodiments of the compound of Formula II, R^{A2}, R^{A3}, R^{A4}, R^{A5} and R^{A6} are H, R^{B2} is H, and R^{B4} is methyl. In some embodiments of the compound of Formula II, R^{A2}, R^{A3}, R^{A4}, R^{A5} and R^{A6} are H, R^{B2} is H, and R^{B4} is methyl. In some embodiments of the compound of Formula II, R^{A2}, R^{A4}, R^{A5} and R^{A6} are H, R^{A3} is methoxy, R^{B2} is H, and R^{B4} is methyl. In some embodiments of the compound of Formula II, R^{A2}, R^{A4}, R^{A5} and R^{A6} are H, R^{A3} is methoxy, R^{B2} is methyl, and R^{B4} is H. In some embodiments of the compound of Formula II, R^{A2}, R^{A3}, R^{A4}, R^{A5} and R^{A6} are H, R^{B2} is methyl, and R^{B4} is H. In some embodiments of the compound of Formula II, R^{A2}, R^{A3}, R^{A5} and R^{A6} are H, R^{A4} is a carboxyl, R^{B2} is methyl, and R^{B4} is methyl. In some embodiments of the compound of Formula II, R^{A2}, R^{A4}, R^{A5} and R^{A6} are H, R^{A3} is a carboxyl, R^{B2} is methyl, and R^{B4} is methyl.

In some embodiments, a compound for use herein comprises the structure of Formula VI;

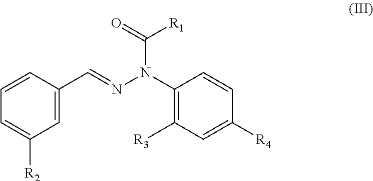

or a pharmaceutically acceptable salt, stereoisomer or tautomer thereof, where R₁ is methyl, fluoromethyl, difluoromethyl, trifluoromethyl, bromomethyl, dibromomethyl or tribromomethyl; R₂ is methyl, methoxy, hydroxyl, halogen, CF₃, OCH₃, OCF₃ or OCBr₃; and R₃ and R₄ are independently selected from hydrogen, hydroxyl, a halogen (e.g., Cl, F or Br), methyl, a methoxy, and an amine. In some embodiments of Formula III, R₁ is CF₃ (trifluoromethyl), R₂ is OCH₃, and R₃ and R₄ are methyl. In some embodiments of Formula III, R₁ is CF₃ (trifluoromethyl), R₂ is OCF₃, and R₃ and R₄ are methyl

In some embodiments, a compound for use herein comprises the structure of Formula IV below, or a pharmaceutically acceptable salt, stereoisomer or tautomer thereof.

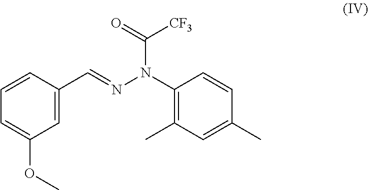 [SMILES: COc1cccc(/C=N/N(C(=O)C(F)(F)F)c2ccc(C)cc2C)c1]

The structure of Formula IV is sometimes referred to herein as “J147”.

The following terms have the respective definitions set out below.

“Alkyl” refers to straight or branched chain alkyl radicals having in the range of about 1 up to about 12 carbon atoms (e.g., methyl, ethyl, propyl, butyl, and the like). “Substituted alkyl” refers to alkyl further bearing one or more substituents (e.g., 1, 2, 3, 4, or even 5) as set forth herein. “Optionally substituted alkyl” refers to alkyl or substituted alkyl.

“Cycloalkyl” refers to cyclic ring-containing groups containing in the range of about 3 up to about 12 carbon atoms. “Substituted cycloalkyl” refers to cycloalkyl further bearing one or more substituents (e.g., 1, 2, 3, 4, or even 5) selected from alkyl, substituted alkyl, as well as any of the substituents set forth herein. “Optionally substituted cycloalkyl” refers to cycloalkyl or substituted cycloalkyl.

“Heterocycle,” “heterocyclic” and like terms refer to cyclic (i.e., ring-containing) groups containing one or more heteroatoms (e.g., N, O, S, or the like) as part of the ring, and having in the range of 1 up to about 14 carbon atoms. “Substituted heterocyclic” and like terms refer to heterocycle further bearing one or more substituents (e.g., 1, 2, 3, 4, or even 5) as set forth herein. Exemplary heterocyclic moieties include saturated rings, unsaturated rings, and aromatic heteroatom-containing ring systems, e.g., epoxy, tetrahydrofuran, oxazoline, pyrrole, pyridine, furan, and the like. “Optionally substituted heterocycle” and like terms refer to heterocycle or substituted heterocycle.

Reference to “optionally substituted bicyclic ring” refers to a bicyclic ring structure as known in the art, optionally including substitutions as defined herein.

“Alkenyl” refers to straight, branched chain, or cyclic hydrocarbyl groups including from 2 to about 20 carbon atoms having at least one, 1-3, 1-2, or one, carbon to carbon double bond. “Substituted alkenyl” refers to alkenyl substituted at 1 or more, e.g., 1, 2, 3, 4, or even 5 positions, with substitution as described herein. “Optionally substituted alkenyl” refers to alkenyl or substituted alkenyl. In some embodiments, an alkenyl is ethylenyl or propylenyl. In certain embodiments, a substituted alkenyl is a substituted ethylenyl or substituted propylenyl. In some embodiments, ethylenyl or propylenyl is substituted with one or more CN moieties. For example, in some embodiments, a substituted ethylenyl comprises (CN)₂C═CH—.

“Aryl” refers to aromatic groups having in the range of 6 up to about 14 carbon atoms. “Substituted aryl” refers to aryl radicals further bearing one or more substituents (e.g., 1, 2, 3, 4, or even 5) selected from alkyl, substituted alkyl, cycloalkyl, substituted cycloalkyl, hydroxyl, alkoxy, aryloxy, mercapto, alkylthio, arylthio, carbonyl, aryl, substituted aryl, heterocyclic, substituted heterocyclic, halogen, trifluoromethyl, pentafluoroethyl, cyano, cyanoalkyl, nitro, amino, amido, amidino, carboxyl, carbamate, SO₂X, wherein X is H, R, NH₂, NHR or NR₂, SO₃Y, wherein Y is H, NH₂, NHR or NR₂, or C(O)Z, wherein Z is OH, OR, NH₂, NHR or NR₂, and the like. “Optionally substituted aryl” refers to aryl or substituted aryl.

“Aralkyl” refers to an alkyl group substituted by an aryl group. “Substituted aralkyl” refers to aralkyl further bearing one or more substituents (e.g., 1, 2, 3, 4, or even 5) selected from alkyl, substituted alkyl, cycloalkyl, substituted cycloalkyl, as well as any of the substituents set forth herein. Thus, aralkyl groups include benzyl, diphenylmethyl, and 1-phenylethyl (—CH(C₆H₅)(CH₃)) among others. “Optionally substituted aralkyl” refers to aralkyl or substituted aralkyl.

“Heteroaryl” refers to aromatic groups containing one or more heteroatoms (e.g., N, O, S, or the like) as part of the aromatic ring, typically having in the range of 2 up to about 14 carbon atoms, and “substituted heteroaryl” refers to heteroaryl radicals further bearing one or more substituents (e.g., 1, 2, 3, 4, or even 5) selected from alkyl, substituted alkyl, cycloalkyl, substituted cycloalkyl, as well as any of the substituents set forth above.

“Heteroaralkyl” and “heteroarylalkyl” refer to an alkyl group substituted by one or more heteroaryl groups. “Substituted heteroaralkyl” refers to heteroaralkyl further bearing one or more substituents (e.g., 1, 2, 3, 4, or even 5) selected from alkyl, substituted alkyl, cycloalkyl, substituted cycloalkyl, as well as any of the substituents set forth herein. “Optionally substituted heteroaralkyl” refers to heteroaralkyl or substituted heteroaralkyl.

“Halogen” and “halo” refer to fluorine, chlorine, bromine or iodine.

“Hydroxyl” and “hydroxy” refer to the functionality —OH.

“Alkoxy” denotes the group —OR, where R is alkyl. “Substituted alkoxy” denotes the group —OR, where R is substituted alkyl. “Optionally substituted alkoxy” refers to alkoxy or substituted alkoxy.

“Aryloxy” denotes the group —OR, where R is aryl. “Substituted aryloxy” denotes the group —OR, where R is substituted aryl. “Optionally substituted aryloxy” refers to aryloxy or substituted aryloxy.

“Mercapto” and “thiol” refer to the functionality —SH.

“Alkylthio” and “thioalkoxy” refer to the group —SR, —S(0)ₙ₌₁₋₂-R, where R is alkyl. “Substituted alkylthio” and “substituted thioalkoxy” refers to the group —SR, —S(O)ₙ₌₁₋₂—R, where R is substituted alkyl. “Optionally substituted alkylthio” and “optionally substituted thioalkoxy” refers to alkylthio or substituted alkylthio.

“Arylthio” denotes the group —SR, where R is aryl. “Substituted arylthio” denotes the group —SR, where R is substituted aryl. “Optionally substituted arylthio” refers to arylthio or substituted arylthio.

“Amino” refers to unsubstituted, monosubstituted and disubstituted amino groups, including the substituent —NH₂, “monoalkylamino,” which refers to a substituent having structure —NHR, wherein R is alkyl or substituted alkyl, and “dialkylamino,” which refers to a substituent of the structure —NR₂, wherein each R is independently alkyl or substituted alkyl.

“Amidino” denotes the group —C(═NR^{q})NR^{r}R^{s}, wherein R^{q}, R^{r}, and R^{s} are independently hydrogen or optionally substituted alkyl.

Reference to “amide group” embraces substituents of the structure —C(O)—NR₂, wherein each R is independently H, alkyl, substituted alkyl, aryl or substituted aryl as set forth above. When each R is H, the substituent is also referred to as “carbamoyl” (i.e., a substituent having the structure —C(O)—NH₂). When only one of the R groups is H, the substituent is also referred to as “monoalkylcarbamoyl” (i.e., a substituent having the structure —C(O)—NHR, wherein R is alkyl or substituted alkyl as set forth above) or “arylcarbamoyl” (i.e., a substituent having the structure —C(O)—NH(aryl), wherein aryl is as defined above, including substituted aryl). When neither of the R groups are H, the substituent is also referred to as “di-alkylcarbamoyl” (i.e., a substituent having the structure —C(O)—NR₂, wherein each R is independently alkyl or substituted alkyl as set forth above).

Reference to “carbamate” embraces substituents of the structure —O—C(O)—NR₂, wherein each R is independently H, alkyl, substituted alkyl, aryl or substituted aryl.

Reference to “ester group” embraces substituents of the structure —O—C(O)—OR, wherein each R is independently alkyl, substituted alkyl, aryl or substituted aryl.

“Acyl” refers to groups having the structure —C(O)R, where R is hydrogen, alkyl, aryl, and the like as defined herein. “Substituted acyl” refers to acyl wherein the substituent R is substituted as defined herein. “Optionally substituted acyl” refers to acyl and substituted acyl.

“Cyanoalkyl” refers to the group —R≡N, wherein R is an optionally substituted alkylenyl.

As used here, “substitution” denotes an atom or group of atoms that has been replaced with another atom or group of atoms (i.e., substituent), and includes all levels of substitution, e.g. mono-, di-, tri-, tetra-, penta-, or even hex-substitution, where such substitution is chemically permissible. Substitutions can occur at any chemically accessible position and on any atom, such as substitution(s) on carbon and any heteroatom, such as oxygen, nitrogen, or sulfur. For example, substituted moieties include those where one or more bonds to a hydrogen or carbon atom(s) contained therein are replaced by a bond to non-hydrogen and/or non-carbon atom(s).

Substitutions can include, but are not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and heteroatoms in other groups as well known in the art.

Non-limiting examples of substituents include, without limitation, halogen, —OH, —NH₂, —NO₂, —CN, —C(O)OH, —C(S)OH, —C(O)NH₂, —C(S)NH₂, —S(O)₂NH₂, —NHC(O)NH₂, —NHC(S)NH₂, —NHS(O)₂NH₂, —C(NH)NH₂, —OR, —SR, —OC(O)R, —OC(S)R, —C(O)R, —C(S)R, —C(O)OR, —C(S)OR, —S(O)R, —S(O)₂R, —C(O)NHR, —C(S)NHR, —C(O)NRR, —C(S)NRR, —S(O)₂NHR, —S(O)₂NRR, —C(NR)NHR, —C(NH)NRR, —NHC(O)R, —NHC(S)R, —NRC(O)R, —NRC(S)R, —NHS(O)₂R, —NRS(O)₂R, —NHC(O)NHR, —NHC(S)NHR, —NRC(O)NH₂, —NRC(S)NH₂, —NRC(O)NHR, —NRC(S)NHR, —NHC(O)NRR, —NHC(S)NRR, —NRC(O)NRR, —NRC(S)NRR, —NHS(O)₂NHR, —NRS(O)₂NH₂, —NRS(O)₂NHR, —NHS(O)₂NRR, —NRS(O)₂NRR, —NHR, —NRR, where R at each occurrence is independently H, optionally substituted alkyl, optionally substituted aryl, or optionally substituted heteroaryl. Also contemplated is substitution with an optionally substituted hydrocarbyl moiety containing one or more of the following chemical functionalities: —O—, —S—, —NR—, —O—C(O)—, —O—C(O)—O—, —O—C(O)—NR—, —NR—C(O)—, —NR—C(O)—O—, —NR—C(O)—NR—, —S—C(O)—, —S—C(O)—O—, —S—C(O)—NR—, —S(O)—, —S(O)₂—, —O—S(O)₂—, —O—S(O)₂—O, —O—S(O)₂—NR—, —O—S(O)—, —O—S(O)—O—, —O—S(O)—NR—, —O—NR—C(O)—, —O—NR—C(O)—O—, —O—NR—C(O)—NR—, —NR—O—C(O)—, —NR—O—C(O)—O—, —NR—O—C(O)—NR—, —O—NR—C(S)—, —O—NR—C(S)—O—, —O—NR—C(S)—NR—, —NR—O—C(S)—, —NR—O—C(S)—O—, —NR—O—C(S)—NR—, —O—C(S)—, —O—C(S)—O—, —O—C(S)—NR—, —NR—C(S)—, —NR—C(S)—O—, —NR—C(S)—NR—, —S—S(O)₂—, —S—S(O)₂—O—, —S—S(O)₂—NR—, —NR—O—S(O)—, —NR—O—S(O)—O—, —NR—O—S(O)—NR—, —NR—O—S(O)₂—, —NR—O—S(O)₂—NR—, —O—NR—S(O)—, —O—NR—S(O)—O—, —O—NR—S(O)—NR—, —O—NR—S(O)₂—O—, —O—NR—S(O)₂—NR—, —O—NR—S(O)₂—, —O—P(O)R₂—, —S—P(O)R₂—, or —NRP(O)R₂—, where R at each occurrence is independently H, optionally substituted alkyl, optionally substituted aryl, or optionally substituted heteroaryl.

In some embodiments, a compound for use herein includes isomers including stereoisomers (e.g., enantiomer and diastereomers), constitutional isomers, tautomers, conformational isomers, and geometric isomers of a compound disclosed herein.

Exemplary constitutional isomers include for example without limitation, isomers resulting from different connectivity of functionalities forming the compounds disclosed herein, for example, 1-propyl versus 2-propyl substitution, and the like. Constitutional isomers in combination with tautomerization additionally embrace bonding rearrangements involving the migration of double bonds and substituents. For example, tautomerization in combination with a 1-3 pleiotropic hydrogen shift can result in constitutional isomerism.

Exemplary conformational isomers include for example without limitation, isomers produced by rotation about a bond wherein the rotation is hindered to the extent that separable isomers result, as well known in the art.

Exemplary geometrical isomers include double bonds in e.g., the “E” or “Z” configuration, as well known in the art.

Compounds disclosed herein can be readily prepared using a suitable synthetic method.

For example, curcumin can be condensed with phenyl hydrazine by warming to reflux overnight in toluene. Optionally, a catalytic amount of acid (HCl) can be employed. In some embodiments, pure curcumin (vs. technical grade) and freshly distilled phenyl hydrazine can be employed.

As another example, 3-methoxy benzaldehyde can be condensed with 2,4-dimethylphenyl hydrazine in methanol employing standard hydrazone preparation conditions (e.g., heating in the microwave to speed the reaction time). Next, the free NH is acylated with TFAA (trifluoroacetic anhydride) plus catalytic (0.1%) amounts of DMAP (dimethylamino pyridine), THE (tetrahydrofuran) or DCM (dichloromethane).

In some embodiments, CF₃ substituted triazoles can be prepared by 1,3-dipolar cycloaddition between suitable aryltrifluoromethylacetylenes and aryl azides. Regioselectivity can be obtained by utilizing a suitable click chemistry (e.g., see Huisgen R. (1984) 1,3-Dipolar Cycloaddition Chemistry, pp. 1-176, Lodon: Wiley; Padwa (1991) Comprehensive Organic Synthesis, Vol. 4: pp. 1069-1109, Oxford: Pergamon; and Fan & Katritzky (1996) Comprehensive Heterocyclic Chemistry II, Vol. 4: pp. 101-126, Oxford: Pergamon). Additional methods of generating compounds disclosed herein can be found in Lima et al., (2015) Chem. Commun. 51:10784-10796 and Kim et al., (2015) Org. Biomol. Chem. 13:9564-9569.

In some embodiments, a compound for use herein is provided in the form of pharmaceutically acceptable salt. A compound for use herein can be complexed with any suitable inorganic or organic salt using a suitable method. In some embodiments, a salt of a compound for use herein is prepared by reacting the compound with a suitable organic or inorganic acid or base. Non-limiting examples of organic salts contemplated for use herein include methanesulfonate, acetate, oxalate, adipate, alginate, aspartate, valerate, oleate, laurate, borate, benzoate, lactate, phosphate, toluenesulfonate (tosylate), citrate, malate, maleate, fumarate, succinate, tartrate, napsylate, methanesulfonate, 2-naphthalenesulfonate, nicotinate, benzenesulfonate, butyrate, camphorate, camphorsulfonate, cyclopentanepropionate, digluconate, dodecylsulfate, glucoheptanoate, glycerophosphate, heptanoate, hexanoate, undecanoate, 2-hydroxyethanesulfonate, ethane sulfonate, and the like. In some embodiments, inorganic salts can be formed from inorganic acids such as sulfate, bisulfate, hemisulfate, hydrochloride, chlorate, perchlorate, hydrobromide, hydroiodide, and the like. Non-limiting examples of a base salt include ammonium salts; alkali metal salts such as sodium salts, potassium salts, and the like; alkaline earth metal salts such as calcium salts, magnesium salts, and the like; salts with organic bases such as dicyclohexylamine salts, N-methyl-D-glucamine, phenylethylamine, and the like; and salts with amino acids such as arginine, lysine, and the like.

Accordingly, in certain embodiments, a method herein comprises administering a therapeutically effect amount of a compound of Formula I, II, III or IV to a subject.

### Respiratory Distress

Acute Respiratory Distress Syndrome (ARDS) is a life-threatening condition associated with fluid buildup in the lungs (pulmonary edema) that results in an inability to maintain adequate blood oxygen levels (hypoxia or hypoxemia). ARDS can have a variety of underlying causes, non-limiting examples of which include pneumonia, sepsis, viral infection and traumatic injury. Prior to the COVID19 pandemic, there was an estimated 3 million annual cases of ARDS globally, responsible for 10% of all ICU patients. The primary treatment option available for ARDS is mechanical ventilation, in which breathing is assisted by a ventilator that pushes air into the lungs.

Patients who recover from ARDS commonly experience some form of cognitive impairment (Hopkins et al. (1999) American Journal of Respiratory and Critical Care Medicine 160(1):50-56; Sasannejad et al. (2019) Critical Care 23(1): 1-12). Some studies have indicated that ARDS survivors suffer from impairments in memory, attention, concentration, and/or processing speed at rates of 70-100% at the time of hospital discharge, 56-80% after one year, and 20% after five years. Some patients with long-term impairment have shown large drops in performance on standard tests of mental acuity, below the 6th percentile of the normal distribution (Hopkins et al. (2005) American Journal of Respiratory and Critical Care Medicine 171(4):340-347). In addition, ARDS survivors are prone to anxiety and depression (Mikkelsen et al. (2009) Respirology 14(1): 76-82; Mikkelsen et al. (2012) American Journal of Respiratory and Critical Care Medicine 185(12): 1307-1315), and a large percentage demonstrate symptoms of post-traumatic stress disorder (Kapfhammer et al. (2004) The American Journal of Psychiatry 161(1):45-52; Mikkelsen et al., 2012).

Imaging studies have found significant brain atrophy and ventricular enlargement in ARDS survivors relative to matched controls (Hopkins et al., (2006) Brain Injury 20(3):263-271). Patients with pre-existing cognitive decline or brain injury are at increased risk (Sasannejad et al., 2019), but there is not a clear association with age or severity of illness (Herridge et al., 2016). The duration of hypoxemia during acute illness also has been associated with increased risk (Mikkelsen et al., 2012), although experiments in a porcine model of ARDS suggested that cytokine-mediated brain damage arising from lung injury is a greater contributor to hippocampal damage than is hypoxemia. A number of other apparent risk factors have been identified, including duration of hypotension, lower central venous pressure, hyperglycemia, and blood glucose variability (Herridge et al. (2016) Intensive Care Medicine 42(5):725-738). The occurrence and duration of delirium, which is common during and after ARDS treatment, is also a risk factor for long-term cognitive decline (Mikkelsen et al., 2012; Sasannejad et al., 2019).

The physiological mechanisms responsible for the cognitive effects of ARDS are unclear. Several possible mechanisms have been suggested. For example, cytokine-induced breakdown of the blood-brain barrier has been suggested as a central event in the process, potentially disrupting the clearance of amyloid-β resulting in further neurological damage. Hippocampal cytokine production has been implicated in depletion of brain-derived neurotrophic factor (BDNF), which is essential for neural plasticity, learning and memory.

Mechanical ventilation used in the treatment of ARDS has significant risks associated with its use (Wolthius et al. (2009) Critical Care (London, England) 13(1): 1-11; Matthay et al. (2019) Nature Reviews. Disease Primers 5(1): 18), and could be an independent contributor to the cognitive impairments experienced by patients after recovery (Bilotta et al. (2019) Critical Care 23(1): 1-3).

There is a lack of established preventative measures or treatments for minimizing ARDS-associated cognitive impairment. Current recommendations are focused on minimizing sedation and tidal volume during ventilator use. There are no pharmaceutical agents currently recommended for prevention or minimization of ARDS-associated cognitive decline. A clinical trial of a statin drug for delirium during treatment and subsequent cognitive impairment failed to show efficacy (Needham et al. (2016) The Lancet Respiratory Medicine 4(3):203-212; clinical trial IDs NCT00979121 and NCT00719446).

As presented herein, pharmaceutical agents with neurotrophic effects, such as those of Formulas I, II, III and IV, can serve to minimize the neurological damage that results from ARDS and its treatment.

Presented herein, in some embodiments, is a method of treating, inhibiting, reducing the severity of, delaying the onset of, or preventing a cognitive impairment or a cognitive disorder resulting from, or caused by respiratory distress in a subject, the method comprising administering a therapeutically effective amount of a compound having the structure of Formula I, II, III or IV to the subject who is at risk of having respiratory distress, who has or is experiencing respiratory distress, or who has previously experienced respiratory distress. A subject who previously experienced respiratory distress is a subject who had or was diagnosed with respiratory distress within 1 day to 10 years of conducting a method herein. In some embodiments, administering a therapeutically effective amount of a compound disclosed herein to a subject who has previously experienced respiratory distress comprises administering a compound disclosed herein to the subject within a time period of 1 day to 10 years after the subject had or was diagnosed with respiratory distress. In certain embodiments, a method comprises preventing or inhibiting a decline of, and/or preventing or inhibiting a worsening of, a pre-existing cognitive impairment, a pre-existing cognitive disorder or a pre-existing neurodegenerative disease, the method comprising administering a therapeutically effective amount of a compound having the structure of Formula I, II, III or IV to the subject who is at risk of having respiratory distress, who has or is experiencing respiratory distress, or who has previously experienced respiratory distress. The term “pre-existing” in the context of respiratory distress means prior to a subject experiencing or having respiratory distress. In some embodiments, a method comprises treating a subject who is at risk of having respiratory distress, who has or is experiencing respiratory distress, or who has previously experienced respiratory distress comprising administering a therapeutically effective amount of a compound having the structure of Formula I, II, III or IV to the subject, wherein a cognitive impairment or a cognitive disorder resulting from, caused by or worsened by the respiratory distress is prevented, ameliorated, inhibited, reduced in severity, or delayed. In certain embodiments, a method comprises treating a subject who is at risk of having respiratory distress, who has or is experiencing respiratory distress, or who has previously experienced respiratory distress comprising administering a therapeutically effective amount of a compound having the structure of Formula I, II, III or IV to the subject, wherein a pre-existing cognitive impairment, a pre-existing cognitive disorder, or a pre-existing neurodegenerative disease is preventing from worsening, or preventing or inhibited from increasing in severity.

Non-limiting examples of respiratory distress include acute respiratory distress, acute respiratory distress syndrome (ARDS), and severe acute respiratory syndrome (SARS). In certain embodiments, respiratory distress is associated with hypoxia. In some embodiments, ARDS is associated with or caused by sepsis, pneumonia, a lung infection (e.g., a fungal infection, a viral infection (e.g., influenza or a coronavirus infection), or a bacterial infection), pancreatitis, physical trauma (e.g., a head injury, chest injury or lung injury), aspiration, smoke inhalation, toxic substance inhalation, idiopathic pulmonary fibrosis, blood transfusion, massive blood transfusion, burns, near-drowning, reactions to medications, drug overdose, shock, lung surgery, cardiopulmonary bypass surgery, disseminated intravascular coagulation, or tick-born relapsing fever. In some embodiments, a coronavirus is SARS-associated coronavirus or SARS-associated coronavirus-2. Accordingly, in certain embodiments, a method herein comprises treating, inhibiting, reducing the severity of, delaying the onset of, or preventing a cognitive impairment or a cognitive disorder resulting from, or caused by ARDS, wherein the ARDS is caused by, or is associated with sepsis, pneumonia, a lung infection (e.g., a fungal infection, a viral infection (e.g., influenza or a coronavirus infection), or a bacterial infection), pancreatitis, physical trauma (e.g., a head injury, chest injury or lung injury), aspiration, smoke inhalation, toxic substance inhalation, idiopathic pulmonary fibrosis, blood transfusion, massive blood transfusion, burns, near-drowning, reactions to medications, drug overdose, shock, lung surgery, cardiopulmonary bypass surgery, disseminated intravascular coagulation, or tick-born relapsing fever.

Non-limiting examples of cognitive impairments and cognitive disorders induced by, worsened by or associated with respiratory distress include loss of memory (short term and long term), loss of concentration, difficulty concentrating, loss of attention, delirium, a confusional state, reduced awareness, difficulty completing familiar or routine tasks; space and time confusion; impairment of vision, color or sign recognition loss, loss of or impairment of communication ability (e.g., speaking difficulty (e.g., slurred, thick or irregular speech), writing difficulty, loss of reading comprehension, vocabulary loss, the like, or combinations thereof), loss of judgment, moodiness, unusual or frequent irritability, loss of or impairment of executive function, depression, anxiety, post-traumatic stress disorder, the like, and combinations thereof. Additional non-limiting examples of cognitive impairments and cognitive disorders induced by, worsened by or associated with respiratory distress include: fatigue (e.g., excessive fatigue); passivity; lethargy; inertia; tremors; ataxia; twitching, atrophy or weakness; shortness of breath; breathing difficulty; depth perception loss; unusual or frequent aggression; paranoia; delusions; withdrawal from social engagement; unusual or frequent stiffness or rigidity; loss of fine or gross motor control; slowing of movement; impaired balance; body instability; posture or gait abnormality (e.g., shuffling walk, unsteady or irregular gait); reduced coordination; motor dysfunction; jerky or involuntary body movement; slowed saccadic eye movement; seizures; dysphagia; difficulty chewing, eating, or swallowing; deterioration in cognition/mental capabilities; dementia; irregular sleep; insomnia; sleep disruption; diagnosed behavioral or psychiatric abnormalities; impaired regulation of social conduct; social withdrawal; over-activity; pacing; wandering; loss of balance; lunging forward when mobilizing; fast walking; imbalance; falls; changes in personality; loss of inhibition or ability to organize information; opthalmoparesis or impaired eye movement; impaired eyelid function; involuntary facial muscle contracture; parkinsonism; the like and combinations thereof. Accordingly, in some embodiments, a method comprises preventing, reducing the severity of, delaying the onset of and/or treating one or more cognitive disorders or cognitive impairments induced by, worsened by or associated with respiratory distress, which methods comprise administering a therapeutically effective amount of a compound disclosed herein, or a pharmaceutical composition comprising a therapeutically effective amount of a compound disclosed herein, to a subject who has, is suspected of having or is at risk of having respiratory distress.

In some embodiments, a method comprises treating, inhibiting, reducing the severity of, delaying the onset of, or preventing memory loss resulting from, or caused by respiratory distress in a subject.

In some embodiments, a method comprises treating, inhibiting, reducing the severity of, delaying the onset of, or preventing impairment of or loss of attention or concentration resulting from, or caused by respiratory distress in a subject.

In some embodiments, a method comprises treating, inhibiting, reducing the severity of, delaying the onset of, or preventing a decline of or a worsening of delirium resulting from, or caused by respiratory distress in a subject.

In some embodiments, a method comprises treating, inhibiting, reducing the severity of, delaying the onset of, or preventing a decline of or a worsening of depression, anxiety and/or post-traumatic stress disorder resulting from, or caused by respiratory distress in a subject.

In some embodiments, a method comprises treating, inhibiting, reducing the severity of, delaying the onset of, or preventing an impairment of or loss of executive function, speech, language or communication ability resulting from, or caused by respiratory distress in a subject.

In some embodiments, a method comprises treating, inhibiting, reducing the severity of, delaying the onset of, or preventing an impairment of or loss of visual and spatial abilities resulting from, or caused by respiratory distress in a subject.

In some embodiments, a method comprises treating, inhibiting, reducing the severity of, delaying the onset of, or preventing an akinetic crisis resulting from, or caused by respiratory distress in a subject.

In some embodiments, a cognitive impairment or cognitive disorder resulting from respiratory distress is detected and/or diagnosed by a medical professional. A cognitive impairment, cognitive disorder or loss of a cognitive function resulting from respiratory distress can be detected and/or diagnosed by comparing the results of a suitable cognitive test (e.g., a psychometric test) conducted before and/or after respiratory distress. A suitable cognitive test can be performed before, and/or at least 1 day, or at least at least 1 week after respiratory distress. In some embodiments, a cognitive test is performed at 1 day to 30 days, 1 day to 15 days, or 1 day to 7 days after a stay in an ICU, after respiratory distress. In some embodiments, a cognitive test is performed at 1 to 6 months, 1 day to 30 days, 1 day to 15 days, or 1 day to 7 days prior to respiratory distress.

In certain embodiments, a subject is not diagnosed with a cognitive disorder or neurodegenerative disease prior to the respiratory distress. In certain embodiments, a subject is not previously diagnosed with, and/or does not have cancer, diabetes, arthritis, insulinoma, stroke, or ischemia (e.g., heart ischemia) prior to respiratory distress.

In certain embodiments, a subject is diagnosed with a cognitive disorder or neurodegenerative disease during or prior to a respiratory distress. In certain embodiments, a subject is previously diagnosed with, has or is suspected of having cancer, diabetes, hyperglycemia, hypoglycemia, fluctuation of serum glucose, in-house acute stress syndrome, delirium, arthritis, pancreatitis and/or insulinoma during or prior to a respiratory distress. In certain embodiments, a subject has or has been diagnosed with asthma during or prior to a respiratory distress. In certain embodiments, a subject or has or has been diagnosed with pneumonia during or prior to a respiratory distress.

### PICCD

Presented herein are methods of preventing, reducing the severity of, delaying the onset of and/or treating PICCD which methods, in certain embodiments, comprise administering a therapeutically effective amount of a compound disclosed herein, or a pharmaceutical composition comprising a compound disclosed herein, to a subject who has, is suspected of having or is at risk of having PICCD. In some embodiments, a method comprises treating a subject who has, is suspected of having or is at risk of having PICCD comprising administering a therapeutically effective amount of a compound disclosed herein, or a pharmaceutical composition comprising a compound disclosed herein, to the subject (e.g., a subject in need thereof).

PICCD is a cognitive condition induced by, caused by or worsened by a stay in an intensive care unit (ICU), intubation and/or connection to a ventilator. In certain embodiments, PICCD is a cognitive impairment, a cognitive disorder and/or a decline of, loss of, or impairment of one or more cognitive functions arising during or after a stay in an intensive care unit (ICU), during or after intubation and/or during or after connection to a ventilator. In some embodiments, PICCD is a new cognitive impairment or cognitive disorder detected or diagnosed after a stay in an ICU, intubation or connection to a ventilator. Accordingly, in certain embodiments, PICCD is a cognitive impairment or cognitive disorder that is not present prior to a stay in an ICU, prior to intubation or prior to connection to a ventilator, but is present after a stay in an ICU, after intubation and/or after connection of a subject to a ventilator. In some embodiments, PICCD comprises or consists of post-intensive care syndrome (Inoue, et al. (2019) Acute Medicine & Surgery 6:233-246).

In some embodiments, PICCD is a chronic condition. In some embodiments, chronic PICCD may last for more than 6 months, more than 12 months or more than 1 year, or more than 5 years in the absence of treatment. Accordingly, in some embodiments, a method comprises preventing or treating chronic PICCD in a subject comprising administering to the subject a therapeutically effective amount of a compound disclosed herein.

In some embodiments, PICCD is an acute condition. In some embodiments, acute PICCD may last up to 6 months, up to 12 months, up to 20 months, or up to 36 months following a stay in an ICU, intubation or connection to a ventilator. Accordingly, in some embodiments, a method comprises preventing or treating acute PICCD in a subject comprising administering to the subject a therapeutically effective amount of a compound disclosed herein.

Non-limiting examples of cognitive impairments and cognitive disorders associated with PICCD include loss of memory (short term and long term), loss of concentration, difficulty concentrating, loss of attention, delirium, a confusional state, reduced awareness, difficulty completing familiar or routine tasks; space and time confusion; impairment of vision, color or sign recognition loss, loss of or impairment of communication ability (e.g., speaking difficulty (e.g., slurred, thick or irregular speech), writing difficulty, loss of reading comprehension, vocabulary loss, the like, or combinations thereof), loss of judgment, moodiness, unusual or frequent irritability, loss of or impairment of executive function, depression, anxiety, post-traumatic stress disorder, the like, and combinations thereof. Additional non-limiting examples of cognitive impairments and cognitive disorders associated with PICCD include: fatigue (e.g., excessive fatigue); passivity; lethargy; inertia; tremors; ataxia; twitching, atrophy or weakness; shortness of breath; breathing difficulty; depth perception loss; unusual or frequent aggression; paranoia; delusions; withdrawal from social engagement; unusual or frequent stiffness or rigidity; loss of fine or gross motor control; slowing of movement; impaired balance; body instability; posture or gait abnormality (e.g., shuffling walk, unsteady or irregular gait); reduced coordination; motor dysfunction; jerky or involuntary body movement; slowed saccadic eye movement; seizures; dysphagia; difficulty chewing, eating, or swallowing; deterioration in cognition/mental capabilities; dementia; irregular sleep; insomnia; sleep disruption; diagnosed behavioral or psychiatric abnormalities; impaired regulation of social conduct; social withdrawal; over-activity; pacing; wandering; loss of balance; lunging forward when mobilizing; fast walking; imbalance; falls; changes in personality; loss of inhibition or ability to organize information; opthalmoparesis or impaired eye movement; impaired eyelid function; involuntary facial muscle contracture; parkinsonism; the like and combinations thereof. Accordingly, in some embodiments, a method comprises preventing, reducing the severity of, delaying the onset of and/or treating one or more cognitive disorders or cognitive impairments of PICCD, which methods comprise administering a therapeutically effective amount of a compound disclosed herein, or a pharmaceutical composition comprising a therapeutically effective amount of a compound disclosed herein, to a subject who has, is suspected of having or is at risk of having PICCD.

In some embodiments, a method of treating or preventing PICCD comprises treating or preventing, delaying the onset of, reducing the severity of, reducing the frequency of, and/or inhibiting an impairment of or loss of memory.

In some embodiments, a method of treating or preventing PICCD comprises treating or preventing, delaying the onset of, reducing the severity of, reducing the frequency of, and/or inhibiting an impairment of or loss of attention or concentration (e.g., a subject's ability to concentrate).

In some embodiments, a method of treating or preventing PICCD comprises treating or preventing, delaying the onset of, reducing the severity of, reducing the frequency of, and/or inhibiting a decline of or a worsening of delirium. Non-limiting examples of symptoms of delirium include reduced awareness of a subject's environment, a decrease in the ability to focus attention, disorientation of time, place and person, language disturbance (e.g., inability to name objects, inability to write, and rambling speech), perceptual disturbances (e.g., hallucinations, illusions or misinterpretations), the like and combinations thereof.

In some embodiments, a method of treating or preventing PICCD comprises treating or preventing, delaying the onset of, reducing the severity of, reducing the frequency of, and/or inhibiting a decline of or a worsening of depression, anxiety and/or post-traumatic stress disorder.

In some embodiments, a method of treating or preventing PICCD comprises treating or preventing, delaying the onset of, reducing the severity of, reducing the frequency of, and/or inhibiting an impairment of or loss of executive function, speech, language or communication ability.

In some embodiments, a method of treating or preventing PICCD comprises treating or preventing, delaying the onset of, reducing the severity of, reducing the frequency of, and/or inhibiting an impairment of or loss of visual and spatial abilities.

In some embodiments, a method of treating or preventing PICCD comprises preventing, reducing the symptoms of, delaying the onset of or treating an akinetic crisis resulting from a stay in an intensive care unit (ICU), intubation and/or during or after connection to a ventilator. An akinetic crisis (also known as acute akinesia), refers to a situation when motor symptoms of Parkinson's Disease (PD) have acutely worsened up to a point when the patient is nearly completely akinetic. Non-limiting examples of symptoms of akinetic crisis also include worsening of, or a presentation of dysphagia, hyperthermia, dysautonomia, tremors, bradykinesia, muscle rigidity, loss of movement, difficulty with bodily movements, slowed movement, impaired posture and balance, speech changes, writing changes, micrographia, stiff muscles, difficulty standing, difficulty walking, involuntary movements, problems with coordination, rhythmic muscle contractions, increased levels of serum muscle enzymes, the like and combinations thereof.

In certain embodiments, PICCD is an exacerbation of or worsening of a pre-existing cognitive disorder, pre-existing cognitive impairment or pre-existing neurodegenerative disease occurring during or after a stay in an intensive care unit (ICU), during or after intubation and/or during or after connection to a ventilator. In some embodiments, PICCD is a pre-existing cognitive disorder or neurodegenerative disease that is exacerbated, increased in severity, or made worse as a result of a stay in an ICU, intubation or connection to a ventilator. The term “pre-existing” in the context of PICCD means prior to admission to an ICU, prior to intubation, and/or prior to connection to a ventilator. Non-limiting examples of a neurodegenerative disease includes Alzheimer's disease, Parkinson's disease, Huntington's disease, amyotrophic lateral sclerosis, glaucoma, retinal degeneration, macular degeneration, age-related hearing loss, mild cognitive impairment, dementia, delirium, depression, anxiety, progressive supranuclear palsy, spinocerebellar ataxia, retinal neuropathy, peripheral neuropathy, diabetic neuropathy, background neuropathy, familial amyloid polyneuropathy, systemic senile amyloidosis, prion disease, scrapie, bovine spongiform encephalopathy, Creutzfeldt-Jakob disease, Gerstmann-Straussler-Scheinker syndrome, amyloidosis, the like and combinations thereof.

In some embodiments, PICCD is detected and/or diagnosed by a medical professional. PICCD can be detected and/or diagnosed by comparing the results of a suitable cognitive test (e.g., a psychometric test) conducted before and/or after a stay in, or admission to, an intensive care unit (ICU), intubation and/or connection to a ventilator. A suitable cognitive test can be performed before, and/or at least 1 day, or at least at least 1 week after a stay in an ICU, after intubation and/or after connection of a subject to a ventilator. In some embodiments, a cognitive test is performed at 1 day to 30 days, 1 day to 15 days, or 1 day to 7 days after a stay in an ICU, after intubation and/or after connection of a subject to a ventilator. In some embodiments, a cognitive test is performed at 1 to 6 months, 1 day to 30 days, 1 day to 15 days, or 1 day to 7 days prior to a stay in an ICU, prior to intubation and/or connection of a subject to a ventilator.

Multiple cognitive domains can be tested to determine the presence of or severity of a cognitive impairment or cognitive disorder, non-limiting examples of which include tests of learning, memory, attention and concentration. Non-limiting examples of cognitive tests that can be performed to diagnose the presence, absence of, severity of, onset of, or amount of a cognitive impairment or cognitive disorder include the Mini-Mental State Examination (MMSE)(e.g., see Saczynski et al, (2012) N. Engl. J. Med. 367:30-39); the Reliable Change Index (e.g., see Lewis et al., (2006) Acta Anaesthesiol Scand. 50:50-57; and Berger et al., (2015) Anesthesiol Clin. 33(3):517-50); the Rey Auditory Verbal Learning Tests; Trail Making Tests, Parts A & B; the Grooved Peg Board Test; the Digit Span Tests; the Stroop Tests, the Four-Field Tests, Erzigkeit's Short Cognitive Performance Test; a patients self-assessment; as well as a variety of tests disclosed in various clinical trials (e.g., see ClinicalTrials.gov Identifier: NCT0361019, NCT03540433, NCT02265263, NCT02650687, NCT02848599, NCT03084393, NCT03029676 and NCT03635229).

PICCD is not clearly tied to any pathological process and the etiology of PICCD has not been precisely determined. Inflammation may play a role in PICCD. However several immunosuppressive/anti-inflammatory drugs have failed to prevent or treat cognitive dysfunction thought to be induced by, caused by, or worsened by surgery. For example, Magnesium (considered an immunosuppressive agent), administered intravenously during cardiac surgery failed to reduce post-surgical cognitive dysfunction in a clinical trial (e.g., see ClinicalTrials.gov Identifier: NCT00041392). As another example, Pexelizumab, a humanized monoclonal antibody used as an immunosuppressive drug, had no effect on post-surgical cognitive dysfunction after coronary artery bypass graft surgery (e.g., Mathew et al. (2004) Stroke 35:2335-239). As yet another example, Minocycline, an antibiotic shown to have anti-inflammatory properties and neuroprotective effects, exacerbated post-surgical cognitive dysfunction (Li W, et al., (2018) J Int Med Res. 46(4): 1404-1413). Accordingly, one cannot predict with any reasonable certainty that PICCD is a disorder caused by inflammation or that a particular agent that has anti-inflammatory or immunosuppressive properties can be used to prevent or treat PICCD.

Also, several drugs that are used to treat neurodegenerative diseases have failed to prevent or treat post-surgical cognitive dysfunction. For example, donepezil (Aricept), a drug used to treat dementia, memory loss and Alzheimer's disease, had no effect on the overall cognitive index in patients with cognitive decline one year after cardiac surgery (Doraiswamy et al., (2007) Psychopharmacol Bull. 40:54-62). Dexmedetomidine, a sedative suggested for use in treating delirium (MacLaren, et al. (2015) Journal of Intensive Care Medicine. 30 (3): 167-175) also failed to show efficacy for post-surgical cognitive dysfunction (Skvarc et al., (2018) Neurosci. Biobehav. Rev. 84, 116-133). A clinical investigation testing the use of Rivastigmine (Exelon), an AD/Parkinson's drug, for the treatment of post-surgical cognitive dysfunction was terminated because it was found to be too dangerous in seriously ill patients after surgery, and at termination, no efficacy was observed (NCT00835159). Accordingly, one cannot predict with any reasonable certainty that a drug used to successfully treat a neurogenerative disease can also be used to prevent or treat PICCD.

### Subjects

The term “subject” refers to a mammal. Any suitable mammal can be treated by a method or composition described herein. Non-limiting examples of mammals include a human, non-human primate (e.g., ape, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a subject is a non-human primate or a human. In some embodiments a subject is a human. A subject can be any age or at any stage of development (e.g., an adult, teen, child, infant, or a mammal in utero). A subject can be male or female.

In some embodiments, a subject is a subject displaying stable cognitive function (e.g., prior to respiratory distress, prior to a stay in an ICU, prior to intubation and/or prior to connection to a ventilator). In some embodiments a subject has not previously been diagnosed with a cognitive disorder or neurodegenerative disease (e.g., prior to respiratory distress, prior to a stay in an ICU, prior to intubation and/or prior to connection to a ventilator). In some embodiments a subject has not previously been diagnosed with cancer, diabetes, arthritis, insulinoma, stroke, or ischemia (e.g., heart ischemia). In some embodiments a subject was not previously administered a compound selected from any one of Formula I, Formula II, Formula III and Formula IV. In certain embodiments a subject is about to have, is scheduled to have, is having and/or has recently had (e.g., within hours to days) a stay in an ICU, intubation or connection to a ventilator.

In certain embodiments, a subject is at risk of developing a PICCD. In some embodiments, a subject at risk of developing a PICCD is 45 years old or older, 50 years old or older, 55 years old or older, 60 years old or older, 65 years old or older, 70 years old or older, or 75 years old or older. In some embodiments, a subject at risk of developing a PICCD is of an age in a range of 45 years to 100 years, 50 years to 100 years, 55 years to 100 years, 60 years to 100 years, 65 years to 100 years, 70 years to 100 years, or 75 years to 100 years old. In some embodiments, a subject at risk of developing a PICCD is a pediatric subject or pediatric patient. In some embodiments, a subject at risk of developing a PICCD is 18 years of age or less, 16 years of age or less, 13 years of age or less, 10 years of age or less, 8 years of age or less or 5 years of age or less. In some embodiments, a subject at risk of developing a PICCD is of an age in a range of 18 years to 1 week, 18 years to 1 month, 18 years to 6 months, 18 years to 1 year, 16 years to 1 year or 13 years to 1 year old.

In certain embodiments, a subject or a subject at risk of PICCD is a subject having, suspected of having or previously diagnosed with a cognitive disorder or neurodegenerative disease (e.g., prior to intubation, admission to an ICU, or connection to a ventilator). In certain embodiments, a subject or subject at risk of PICCD is a subject having, suspected of having or previously diagnosed with dementia, delirium, depression, anxiety and/or in-hospital acute stress symptoms.

In certain embodiments, a subject or a subject at risk of PICCD is a subject having, suspected of having or previously diagnosed with diabetes, hyperglycemia, hypoglycemia, fluctuation of serum glucose, and/or pancreatitis. In certain embodiments, a subject or a subject at risk of PICCD is a subject having, suspected of having or previously diagnosed with cancer.

Certain studies have suggested that a subject of the female sex is significantly more prone to develop PICCD. Accordingly, in some embodiments, a subject at risk is female.

Certain studies have suggested that patients having secondary health conditions, non-limiting examples of which include organ dysfunction, pulmonary dysfunction and asthma have a higher risk of developing PICCD. Accordingly, in certain embodiments, a subject or a subject at risk of PICCD is a subject having, suspected of having or previously diagnosed with organ dysfunction, pulmonary dysfunction or asthma.

In certain embodiments, a subject at risk of PICCD is a subject with an educational level equivalent to 12^{th} grade (i.e., high school GED or diploma) or less.

In certain embodiments, a subject or a subject at risk of PICCD is a subject having, suspected of having or previously diagnosed with acute respiratory syndrome, severe acute respiratory syndrome, asthma, pneumonia, or an infection. In certain embodiments, a subject or a subject at risk of PICCD is a subject having, suspected of having or previously diagnosed with an infection with a pathogen, non-limiting examples of which include a bacteria, virus or fungus. In certain embodiments, a subject or a subject at risk of PICCD is a subject that is infected with a coronavirus, non-limiting examples of which include SARS-associated coronavirus (SARS-CoV) and SARS-associated coronavirus-2 (SARS-CoV-2). In certain embodiments, a subject or a subject at risk of PICCD is a subject that has, or is suspected of having COVID-19.

### Pharmaceutical Compositions

In some embodiments, a composition or pharmaceutical composition comprises a compound disclosed herein. In some embodiments, a composition or pharmaceutical composition comprises a therapeutically effective amount of a compound disclosed herein. In some embodiments, a composition or pharmaceutical composition comprises a compound disclosed herein in an amount in a range of 1 μg to 100 mg, or 10 μg to 100 μg. In some embodiments provided herein is a pharmaceutical composition comprising a compound disclosed herein for use in conducting a method described herein. In some embodiments, a pharmaceutical composition comprises a compound disclosed herein and a pharmaceutically acceptable excipient, diluent, additive or carrier.

A pharmaceutical composition can be formulated for a suitable route of administration. In some embodiments a pharmaceutical composition is formulated for oral, subcutaneous (s.c.), intradermal, intramuscular, intraperitoneal and/or intravenous (i.v.) administration. In certain embodiments, a pharmaceutical composition contains formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants. In particular, a pharmaceutical composition can comprise any suitable carrier, formulation, or ingredient, the like or combinations thereof as listed in “Remington: The Science And Practice Of Pharmacy” Mack Publishing Co., Easton, Pa., 19^{th} Edition, (1995)(hereafter, Remington '95), or “Remington: The Science And Practice Of Pharmacy”, Pharmaceutical Press, Easton, Pa., 22^{nd} Edition, (2013)(hereafter, Remington 2013), the contents of which are incorporated herein by reference in their entirety.

In certain embodiments, a pharmaceutical composition comprises a suitable excipient, non-limiting examples of which include anti-adherents (e.g., magnesium stearate), a binder, fillers, monosaccharides, disaccharides, other carbohydrates (e.g., glucose, mannose or dextrin), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington '95 or Remington 2013. The term “binder” as used herein refers to a compound or ingredient that helps keeps a pharmaceutical mixture combined. Suitable binders for making pharmaceutical formulations and are often used in the preparation of pharmaceutical tablets, capsules and granules are known to those skilled in the art.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent include those having a sulfhydryl group (e.g., a thiol) such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non-limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition, pharmaceutical composition or compound disclosed herein is substantially free of contaminants (e.g., blood cells, platelets, polypeptides, minerals, blood-borne compounds or chemicals, virus, bacteria, other pathogens, toxin, and the like). In some embodiments a composition, pharmaceutical composition or compound disclosed herein is substantially free of serum and serum contaminants (e.g., serum proteins, serum lipids, serum carbohydrates, serum antigens and the like). In some embodiments a composition, pharmaceutical composition or compound disclosed herein is substantially free of a pathogen (e.g., a virus, parasite or bacteria). In some embodiments a composition, pharmaceutical composition or compound disclosed herein is substantially free of endotoxin. In some embodiments a composition, pharmaceutical composition or compound disclosed herein is sterile. In certain embodiments, a composition or pharmaceutical composition disclosed herein comprises a compound of Formula I, II, III or IV.

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parenteral administration may contain one or more excipients. In some embodiments a pharmaceutical composition is lyophilized to a dry powder form. In some embodiments a pharmaceutical composition is lyophilized to a dry powder form, which is suitable for reconstitution with a suitable pharmaceutical solvent (e.g., water, saline, an isotonic buffer solution (e.g., PBS), DMSO, combinations thereof and the like). In certain embodiments, reconstituted forms of a lyophilized pharmaceutical composition are suitable for parenteral administration (e.g., intravenous administration) to a mammal.

In certain embodiments, a pharmaceutical composition is configured for oral administration and may be formulated as a tablet, microtablet, minitablets, micropellets, powder, granules, capsules (e.g., capsules filled with microtablets, micropellets, powders or granules), emulsions, solutions, the like or combinations thereof. Pharmaceutical compositions configured for oral administration may comprise suitable coatings to delay or sustain release of the active ingredient, non-limiting examples of which include enteric coatings such as fatty acids, waxes, shellac, plastics, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, cellulose acetate trimellitate, sodium alginate, zein, plant fibers, the like and combinations thereof.

In some embodiments a pharmaceutical compositions described herein may be configured for topical administration and may include one or more of a binding and/or lubricating agent, polymeric glycols, gelatins, cocoa-butter or other suitable waxes or fats. In some embodiments a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a compound using a topical patch.

In certain embodiments, an optimal pharmaceutical composition is determined by one skilled in the art depending upon, for example, on the intended route of administration, delivery format and desired dosage (see e.g., Remington '95 or Remington 2013, supra). A pharmaceutical composition can be manufactured by any suitable manner, including, e.g., by means of conventional mixing, dissolving, granulating, dragee-making, levigating, emulsifying, encapsulating, entrapping or tableting processes (e.g., see methods described in Remington '95 or Remington 2013).

### Route of Administration

Any suitable method of administering a composition, pharmaceutical composition or compound disclosed herein to a subject can be used. Any suitable formulation and/or route of administration can be used for administration of a compound disclosed herein or composition disclosed herein (e.g., see Fingl et al. 1975, in “The Pharmacological Basis of Therapeutics”, which is incorporated herein by reference in its entirety). A suitable formulation and/or route of administration can be chosen by a medical professional (e.g., a physician) in view of, for example, a subject's risk, age, and/or condition. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavemous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a compound disclosed herein or pharmaceutical composition described herein is administered to the lungs, bronchial passages, trachea, esophagus, sinuses, or nasal passages using a suitable method, non-limiting examples of which include intranasal administration, intratracheal instillation, and oral inhalative administration (e.g., by use of an inhaler, e.g., single/multiple dose dry powder inhalers, nebulizers, and the like).

In some embodiments a compound disclosed herein or a pharmaceutical composition disclosed herein is provided to a subject. For example, a composition that is provided to a subject is sometimes provided to a subject for self-administration or for administration to a subject by another (e.g., a non-medical professional). As another example, a composition can be provided as an instruction written by a medical practitioner that authorizes a patient to be provided a composition or treatment described herein (e.g., a prescription). In yet another example, a composition can be provided to a subject where the subject self-administers a composition orally, intravenously or by way of an inhaler, for example.

Alternately, one can administer a compound disclosed herein or composition in a local rather than systemic manner, for example, via direct application to the skin, mucous membrane or region of interest for treating, including using a depot or sustained release formulation.

In certain embodiments a pharmaceutical composition comprising a compound disclosed herein is administered alone (e.g., as a single active ingredient (AI or e.g., as a single active pharmaceutical ingredient (API)). In other embodiments, a pharmaceutical composition comprising a compound disclosed herein is administered in combination with one or more additional AIs/APIs, for example, as two separate compositions or as a single composition where the one or more additional AIs/APIs are mixed or formulated together with a compound disclosed herein in a pharmaceutical composition.

### Dose and Therapeutically Effective Amount

In some embodiments, an amount of a compound disclosed herein (e.g., in a pharmaceutical composition) is a therapeutically effective amount. In certain embodiments, a pharmaceutical composition comprises a therapeutically effective amount of a compound disclosed herein. In some embodiments, a therapeutically effective amount of a compound disclosed herein is administered to a subject. In some embodiments, a therapeutically effective amount of a compound disclosed herein is an amount needed to obtain an effective therapeutic outcome. In certain embodiments, a therapeutically effective amount of a compound disclosed herein is an amount sufficient to treat or prevent a cognitive impairment or cognitive disorder resulting from respiratory distress. In certain embodiments, a therapeutically effective amount of a compound disclosed herein is an amount sufficient to treat or prevent PICCD. Determination of a therapeutically effective amount is well within the capability of those skilled in the art, especially in light of the detailed disclosure provided herein.

In certain embodiments, a therapeutically effective amount is an amount high enough to provide an effective therapeutic effect (e.g., a beneficial therapeutic effect) and an amount low enough to minimize unwanted adverse reactions. Accordingly, in certain embodiments, a therapeutically effective amount of a compound disclosed herein may vary from subject to subject, often depending on age, weight, general health condition of a subject, severity of a condition being treated, length of a stay in an ICU, duration of intubation or amount of time a subject is connected to a ventilator. Thus, in some embodiments, a therapeutically effective amount is determined empirically. Accordingly, a therapeutically effective amount of a compound that is administered to a subject can be determined by one of ordinary skill in the art based on amounts found effective in animal or clinical studies, a physician's experience, and suggested dose ranges or dosing guidelines, for example.

In certain embodiments, a therapeutically effective amount of a compound disclosed herein is administered at a suitable dose (e.g., at a suitable volume, frequency and/or concentration, which often depends on a subject's weight, age and/or condition) intended to obtain an acceptable therapeutic outcome. In certain embodiments, a therapeutically effective amount of a compound comprises one or more doses selected from at least 0.01 mg/kg (e.g., mg of a compound per kg body weight of a subject), at least 0.1 mg/kg, at least 0.5 mg/kg, at least 1 mg/kg, at least 10 mg/kg or at least 100 mg/kg. In certain embodiments, a therapeutically effective amount of a compound is selected from one or more doses of about 0.001 mg/kg (e.g., mg of a compound per kg body weight of a subject) to about 5000 mg/kg, 0.01 mg/kg to 1000 mg/kg, 0.01 mg/kg to 500 mg/kg, 0.1 mg/kg to 1000 mg/kg, 1 mg/kg to 1000 mg/kg, 10 mg/kg to 1000 mg/kg, 100 mg/kg to 1000 mg/kg, 0.1 mg/kg to 500 mg/kg, 0.1 mg/kg to 250 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg, 0.5 mg/kg to 5 mg/kg, intervening amounts and combinations thereof. In some aspects a therapeutically effective amount of a compound administered to a subject comprises one or more doses of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 50 mg/kg, 100 mg/kg, 500 mg/kg, and intervening amounts and combinations thereof. In some embodiments a therapeutically effective amount of a compound disclosed herein is between about 0.1 mg/kg and about 50 mg/kg.

In certain embodiments, a therapeutically effective amount of a compound comprises one or more doses where the dose amount is determined with reference to a dose selected from a does efficacious for a mouse as follows. A mouse dose of D ug/g=D mg/kg which may be converted by conversion factors known in the art such 3/37 which is a conversion factor that takes into account differences in body surface area between mice and humans. (See FASEB J. 22, 659-661 (2007).) Thus in some embodiments a suitable dose for a human would be (3/37)*D mg/kg based on a mouse dose of D ug/g. For example, a mouse dose of 5 ug/g would convert for a human to 5 mg/kg×3/37=0.40 mg/kg, which for a 60 kg human would result in a 24 mg dose. Using this conversion, (i) for a 10 ug/g mouse dose, the human dose would be 0.81 mg/kg, and (ii) for a 25 ug/g mouse dose, the human dose would be 2.02 mg/kg.

In some aspects a therapeutically effective amount of a compound administered to a subject comprises one or more doses of about 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, and intervening amounts and combinations thereof. In some embodiments a therapeutically effective amount of a compound disclosed herein is between about 0.4 mg/kg and about 2.0 mg/kg.

In some embodiments administering a therapeutically effective amount of a compound disclosed herein, or a pharmaceutical composition comprising a compound disclosed herein, comprises administering a suitable dose at a frequency or interval as needed to obtain an effective therapeutic outcome. In some embodiments administering a therapeutically effective amount of a compound or a pharmaceutical composition disclosed herein comprises administering a suitable dose hourly, every two hours, every 4 hours, every 6 hours, three times a day, twice a day, once a day, six times a week, five times a week, four times a week, three times a week, twice a week, weekly, at combinations thereof, and/or at regular or irregular intervals thereof, and/or simply at a frequency or interval as needed or recommended by a medical professional. In some embodiments, a therapeutically effective amount of a compound or pharmaceutical composition is administered continuously by, for example by intravenous administration.

In some embodiments a therapeutically effective amount of a compound is administered to a subject prior to, during and/or after a stay in an ICU, intubation or connection to a ventilator. In some embodiments a therapeutically effective amount of a compound is administered to a subject up to 3 days prior to, up to 2 days prior to, up to 1 day prior to, up to 20 hours prior to, up to 15 hours prior to, up to 10 hours prior to, up to 5 hours prior to, up to 2 hours prior to or up to 1 hour prior to a stay in an ICU, intubation or connection to a ventilator.

In some embodiments a therapeutically effective amount of a compound is administered to a subject 0 to 72 hours, 0 and 48 hours, 0 to 24 hours, 0 to 12 hours, 0 to 6 hours, 0 to 4 hours, or 0 to 2 hours before a stay in an ICU, intubation or connection to a ventilator. In some embodiments a therapeutically effective amount of a compound is administered during a stay in an ICU, during intubation and/or during a time when a subject is connected to a ventilator. In some embodiments a therapeutically effective amount of a compound is administered intermittently or continuously for up to 1 hour after, 2 hours after, 4 hours after, 6 hours after, 12 hours after, 24 hours after, 2 days after, 3 days after, a week after, 1 month after, 3 months after, 6 months after, 12 months after, 18 months after, 24 months after or up to 36 months after a stay in an ICU, intubation or connection to a ventilator.

The term “connected” as used herein with reference to a ventilator means “operably connected” such that the ventilator supplies oxygen to the lungs of the subject or patient. Ventilators are known in the art and are often medical machines that actively move breathable air and/or oxygen into and out of the lungs of a patient.

The phrase, “Intensive care unit (ICU)” refers to an intensive care unit, intensive therapy unit, intensive treatment unit and/or a critical care unit, which is often a special department or branch of a hospital that provides intensive treatment medicine to patients with severe or life-threatening illnesses and injuries, which often require constant care and close monitoring of life support equipment and medications. ICUs are known in the art and are distinct from standard hospital rooms. ICUs are often staffed by highly trained physicians, nurses and therapists who specialize in caring for critically ill patients. ICUs are also distinguished from general hospital wards by a higher staff-to-patient ratio and to access to advanced medical resources and equipment that is not routinely available. The phrase, “admitted to an intensive care unit”, refers to a subject who occupies, is brought into, or is moved into an intensive care unit of a hospital as a patient.

### Kits

In some embodiments, provided herein is a kit comprising a compound disclosed herein or a pharmaceutical composition comprising a compound disclosed herein. In some embodiments, a kit comprises one or more doses of a pharmaceutical composition comprising a compound disclosed herein. In some embodiments, a kit comprises one or more packs and/or one or more dispensing devices, which can contain one or more doses of a compound disclosed herein, or pharmaceutical composition thereof, as described herein. Non-limiting examples of a pack include a metal, glass, or plastic container, syringe or blister pack that comprises a compound disclosed herein or a composition described herein. In certain embodiments, a kit comprises a dispensing device such as a syringe or inhaler, that may or may not comprise a compound disclosed herein or a composition described herein. A pack and/or dispenser device can be accompanied by instructions for administration. The pack or dispenser can also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

In some embodiments a kit or pack comprises an amount of a compound disclosed herein sufficient to treat a patient for 1 day to 1 year, 1 day to 180 days, 1 day to 120 days, 1 day to 90 days, 1 day to 60 days, 1 day to 30 days, 1-24 hours, 1-12 hours, 1-4 hours, or amount of time there between.

A kit optionally includes a product label and/or one or more packaging inserts, that provide a description of the components or instructions for use in vitro, in vivo, or ex vivo, of the components therein. Exemplary instructions may include instructions for a treatment protocol or therapeutic regimen. In certain embodiments, a kit comprises packaging material, which refers to a physical structure housing components of the kit. The packaging material can maintain the components sterilely and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory-type cards. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. A kit can additionally include labels or instructions for practicing any of the methods described herein. Product labels or inserts can include information on potential adverse side effects and/or warnings.

### EXAMPLES

### Example 1—Prophetic Treatment

A male subject, age 60, is diagnosed with ARDS. The subject is administered J147 intravenously at a dose of 1 mg/kg, followed by administration of daily i.v. doses of 1 mg/kg continuing for a time period of 1 to 6 weeks. The subject is assessed for loss of cognitive function by administering one or more suitable cognitive tests after recovery. After the final test, the subject is determined to have little or no loss of cognitive function.

### Example 2—Prophetic Treatment

A male subject, age 60, is diagnosed with COVID-19 and serve acute respiratory syndrome. Prior to intubation and connection to a ventilator, J147 is administered intravenously at a dose of 1 mg/kg, with multiple subsequent doses administered once a day while connected to the ventilator, followed by administration of daily oral doses of 1 mg/kg starting on the day the subject was removed from the ventilator and continuing for a time period of 1 to 6 weeks. The subject is assessed for PICCD by administering one or more suitable cognitive tests starting at 1 week after removal from the ventilator. After the final test, the subject is determined to have little or no loss of cognitive function.

### Example 3—Demonstration of Neurogenesis and Improvement of Structural and Functional Recovery in a Mouse Model of Neonatal Hypoxic-Ischemic Brain Injury

Nearly one million newborns per year suffer neonatal hypoxic-ischemic brain injury (HI), which results in high rates of severe neurologic injury in survivors. Hypothermic neuroprotection is the only proven therapeutic alternative, but the benefit it provides is very limited and, in many cases, it cannot be instituted in time to achieve any benefit. The primary pathology in neonatal HI is neuronal cell death and dysfunction, and thus preventing neuronal death and enhancing neurogenesis to promote repair is the desired treatment strategy.

The compound of Formula IV, J147, is highly neuroprotective against a broad variety of neurotoxic insults and is neurogenic through endogenous production of brain derived growth factor, a trophic factor that enhances neural stem cell proliferation and differentiation. This mouse model study demonstrated J147's ability to improve outcomes in neonatal HI by strongly protecting against neuronal death and showed its capacity to significantly upregulate neurogenesis. The effectiveness of J147 for treating HI in mice supports the use of J147 for treatment of and/or prevention of the consequences of hypoxia associated with suffering from ARDS or PICCD in view of the hypoxia associated with intubation or connection to a ventilator or respiratory distress or other conditions or treatments having a risk of hypoxia during a stay in an ICU.

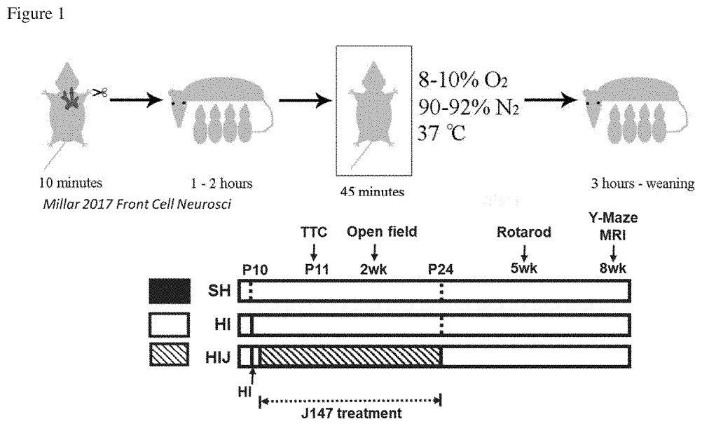

Postnatal day 10 mice were subjected to the modified Vanucci-Rice model of neonatal hypoxic-ischemic injury (permanent right common carotid artery ligation followed by 8% oxygen/balance nitrogen exposure) and then treatment started the same day by daily oral gavage with J147 at 10 mg/kg/day for 2 weeks after injury. Infarction volume, performance on functional tests of coordination, locomotion and memory, markers of neurogenesis, and apoptotic cell death were measured. Experimental design is summarized in FIG. 1.

Hypoxic-ischemic (HI) injury and J147 treatment: To induce HI injury, permanent right common carotid artery ligation was performed in p10 C57BL6 mice followed by 8% oxygen/balance nitrogen exposure for 45 min at 37° C. hypoxic chamber. From the day of HI injury, either J147 (10 mg/kg/day) or vehicle (corn oil, same volume) were once daily administered through oral gavage for a period of two weeks.

TTC staining: 24 h after HI injury, 2 mm coronal brain sections were immersed in 1% 2,3,5-Triphenyltetrazolium chloride (TTC) at 37° C. for 30 min. Images of individual sections were digitized and infarct area was measured with the ImageJ software. Injury volumes were calculated using [contralateral hemisphere volume-(ipsilateral hemisphere volume-lesion volume)]/contralateral hemisphere volume×100.

Magnetic resonance imaging (MRI): Postmortem high resolution T2-weighted MRI of mouse brains at p60 were performed on an 9.4 Tesla vertical bore NMR spectrometer using a 15 mm diameter volume coil as the radio-frequency transmitter and receiver. A 3D rapid acquisition with refocused echoes sequence was used (TE/TR=40/2000 ms, RARE factor=8, 4 signal averages, FOV=16.0 mm×9.0 mm×18.0 mm, matrix size=128×72×144, native resolution≈125×125×125 um3, time=3 h). Volumetric measurements were performed using ROI editor software.

Behavioral Testing: Three different tests which assay different neurologic domains were employed: i) open field test, which measures general locomotor activity, was performed on p14 by measuring escape latency from a 13 cm circle for up to 30 sec; iii) rotarod, which measures motor coordination, was performed on p45 by measuring endurance time on the rotating rod (9.3 cm width, 3 cm diameter) that accelerated 0.3 rpm/sec from 4 to 99 rpm up to 3 min; and iii) Y-maze, which measures spatial learning, was performed on p60 by measuring spontaneous alternations (consecutive triplets of different arm choices) of 3 arm entries for 5 min.

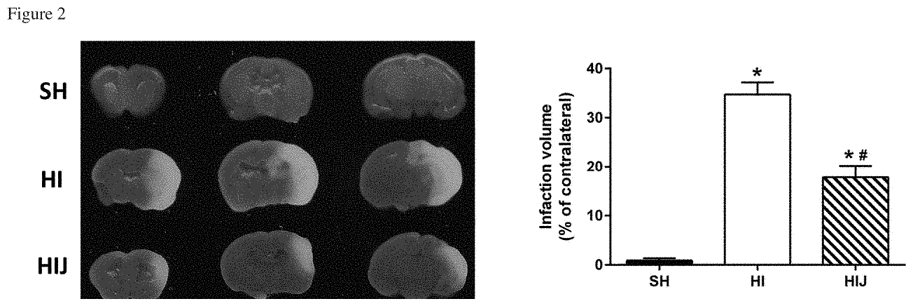

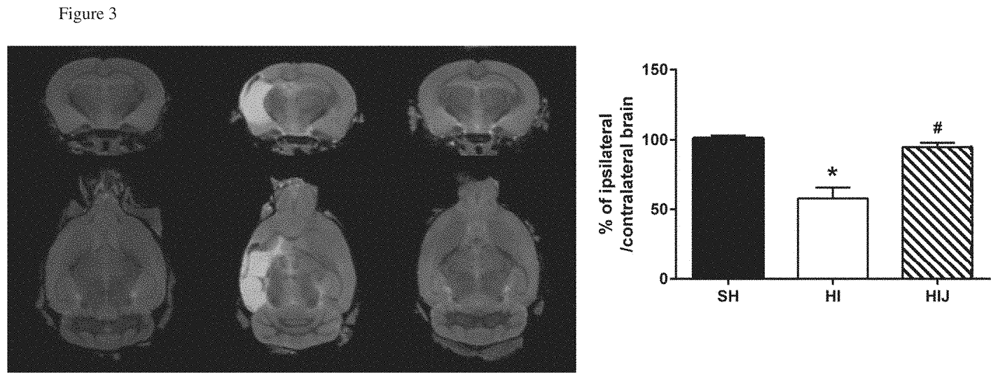

Administration of J147 reduced infarction volume both acutely and chronically. Mice treated with J147 showed reduced lesion size in the hippocampus by histologic measurements at 24 hours and magnetic resonance imaging measurements at 50 days as shown in FIGS. 2 and 3.

FIG. 2 illustrates in Example 3 the 2,3,5-triphenyltetrazolium chloride (TTC) staining of the mouse hippocampus after 24 hour of HI injury in representative brain coronal sections where white indicates damage areas. The sham-treated (SH) sections show no injury, the HI sections show substantial injury and the HIJ Sections from mice treated with J147 show reduced areal extent of injury. MRI measurements at 50 days showed no infarction volume for SH mice and an approximate 50% reduction in infarction volume for HIJ mice compared with untreated HI mice.

FIG. 3 illustrates in Example 3 the effect of treatment with J147 through the measurement of ipsilateral hemisphere volume as a percentage of contralateral hemisphere volume with the degree of reduction from 100% reflecting the magnitude of the infarct volume. SH shows no effect. HIJ shows substantial reduction of effect compared with HI.

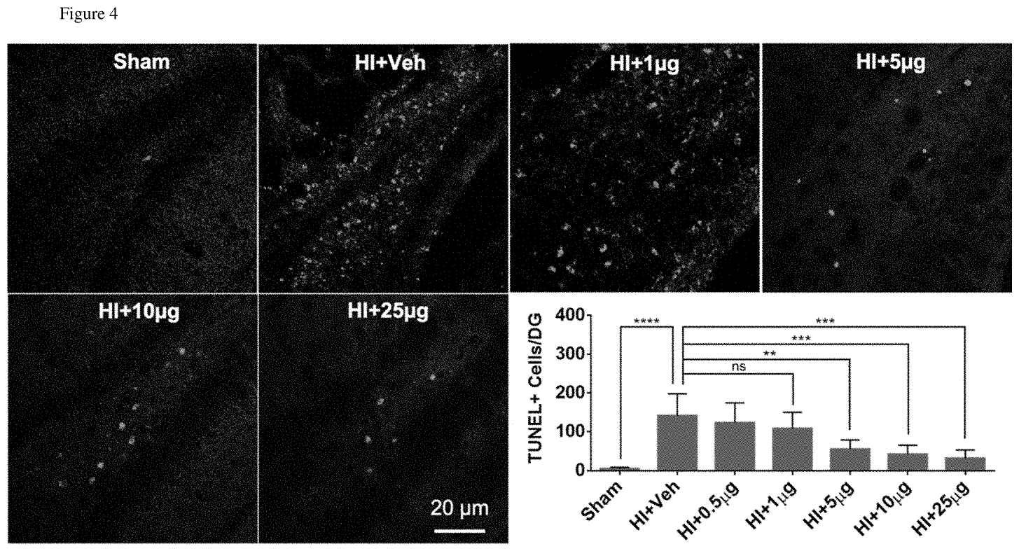

J147 shows dose dependency for the reduction of apoptotic cell death in hippocampus in a neonatal hypoxic-ischemic encephalopathy (HIE) mouse model. C57BL6 mice were subjected to complete unilateral carotid blockage using cauterizer at P10 followed by 45 min hypoxic exposure at 8% O2, and this was followed by daily oral gavage delivery of J147 in a range of doses or vehicle at P10-P14. Then animals were sacrificed and processed for TUNEL staining. In sham group, very few TUNEL+ cells are observed in dentate gyrus (DG, 4.7+4.1/each). HIE dramatically increases the apoptotic cells (141.7+56.6; p<0.0001) and treatment of J147 dose-dependently decreases this number. 123.2+51.5 TUNEL+ cells are detected in 0.5 μg/g/day group and 107.8+42.7 cells in 1 μg/g/day group (p>0.05). 5 μg/g/day J147 significantly reduces the number (55+23.6; p<0.01). For 10 μg/g/day group and 25 μg/g/day group, 42.2+23 (p<0.001) and 31.7+21.4 (p<0.001) cells are found in DG (One-way ANOVA; n=6). Results are shown in FIG. 4.

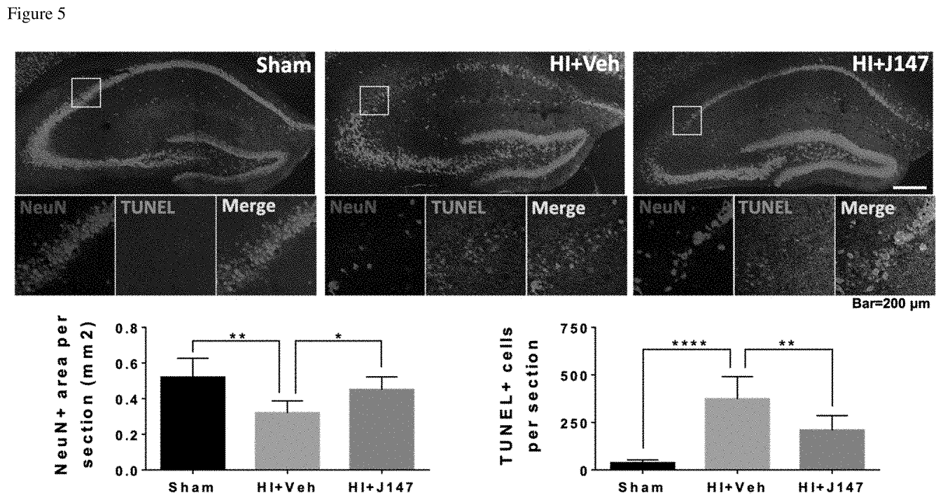

J147 treatment results in a reduction in apoptotic cell death as shown in FIG. 5 through the Measurement of NeuN+ area and counting TUNEL+ cells in hippocampus in sham-treated mice, HI mice and HI mice treated with J147. Neonatal hypoxic-ischemia (HI) was performed at P10 and daily 10 μg/g J147 was gavage-fed from P10 to P14. HI causes NeuN+ area decrease (0.52 mm2/section (SH) vs. 0.32 mm2/section (HI); p=0.0025) and J147 feeding increases the area (0.45 mm2/section (HI+J147); p=0.0433) back closer to the level of the sham-treated mice. HI induces TUNEL+ apoptotic cells (36.33/section (SH) vs. 372.67/section (HI); p<0.0001) and J147 reduces this number (208.17/section (HI+J147); p=0.0092).

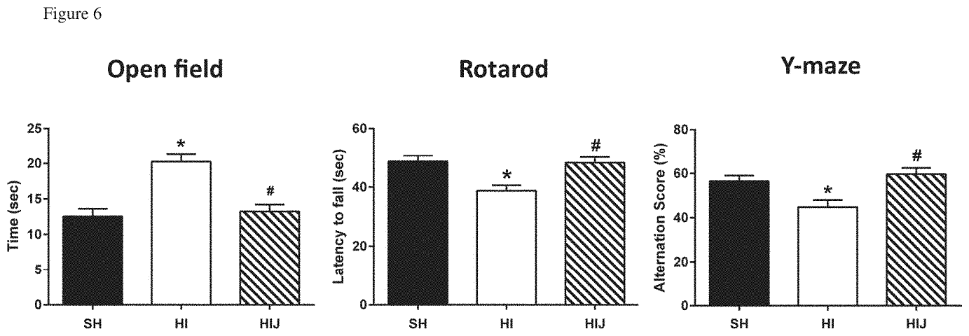

FIG. 6 illustrates in Example 3 the functional recovery of HI mice induced by treatment with J147 in open field, rotarod, and y-maze testing. J147 treated mice showed a significant improvement in performance in the open field test at 4 days post HI, the rotarod test at 5 weeks and the dentate gyrus-dependent y-maze spatial learning task at 50 days (33%, p<0.001 compared to HI without treatment group). In each case, the HI mice performed worse than the sham-treated mice and the performance of the J147 treated mice was comparable to that of the sham-treated mice, i.e., the J147 treated mice showed substantial functional recovery in behavioral testing from the degradation in performance associated with HI injury. Four days after HI injury, mice were given the open field test to evaluate locomotion and pivoting behavior. Five weeks after HI injury, mice were given the rotarod test to evaluate motor coordination. Fifty days after HI injury, mice were given the spontaneous alternation in Y-maze test to assess working memory.

Summary: Infarct size, as measured by TTC staining was reduced by 48% relative to HI controls with one day of J147 treatment (p<0.001) and MRI imaging showed 88% reduction in lesion size (p<0.001) in adult mice with two weeks of J147 treatment. Consistent with that effect, J147 treatment resulted in a roughly 50% reduction in apoptotic cell death in the hippocampus. Furthermore, behavioral testing indicated improvement with J147 treatment. Acute performance on the open field test at 4 days and chronic performance on the rotarod test were significantly improved by about 25% in the J147 treatment group, while y-maze spatial learning task showed 33% improvement compared to HI controls. Taken together, these findings strongly suggest that J147 promotes neurologic recovery after neonatal HI via neuroprotection and upregulation of neurogenesis. With neural precursor marker fluorescence staining, nestin-positive cells were increased at one day and one week after injury with J147 treatment. At one week, there was a significant increase in Tbr2-positive cells, which indicated rapid proliferation of intermediate neuronal precursors. These results support the conclusion that treatment with J147 is protective against hypoxia-induced injuries and promotes recovery from hypoxia-induced injuries and therefore would be effective for PICCD and ARDS as described in certain embodiments of this invention.

References: 1. Patel S D, et al. Biochem Soc Trans. 2014 April; 42(2):564-8; 2. Lai M, et al. J Biomed Biotechnol. 2011: 609813; 3. Prior M, et al. Alzheimers Res Ther. 2013 May 14; 5(3):25; 4. Chen Q, et al. PLoS One. 2011; 6(12): e27865; 5. Nicola Z, et al. Front Neuroanat. 2015 May 7; 9:53.

### Example 4—Certain Non-Limiting Embodiments

Example 4 consists of the following embodiments A1-A5, B1, C1-C27, D1-D2 as set below in this paragraph:

A1. A method for treating or preventing post-intensive care cognitive dysfunction (PICCD) in a subject in need thereof comprising administering a therapeutically effective amount of a compound having the structure of Formula I:

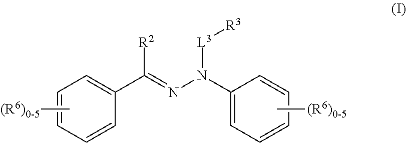

or a pharmaceutically acceptable salt, stereoisomer or tautomer thereof,
wherein:
   R² is selected from the group consisting of H and methyl;
   R³ is trifluoromethyl or other fluoro substituted alkyl;
   L³ is a carbonyl; and
   R⁶ at each occurrence is independently selected from the group consisting of alkyl, substituted alkyl, cycloalkyl, substituted cycloalkyl, hydroxyl, alkoxy, substituted alkoxy, aryloxy, substituted aryloxy, mercapto, alkylthio, arylthio, carbonyl, aryl, substituted aryl, substituted heterocyclic, halogen, cyano, cyanoalkyl, nitro, amino, amidino, carbamate, S(O)ₙR⁷ and C(O)R⁸ or two R⁶ at adjacent positions combine to form an optionally substituted heteroaryl or heteroalkyl ring fused with the adjoining phenyl moiety;
   R⁷ is H, R⁹, NH₂, HNR⁹ or NR⁹R¹⁰;
   R⁸ is OH, OR⁹, NH₂, NHR⁹ or NR⁹R¹⁰;
   R⁹ and R¹⁰ at each occurrence are independently optionally substituted alkyl; and
   n=1 or 2.
      A2. The method of embodiment A1, wherein R⁶ at each occurrence is selected from the group consisting of alkyl, substituted alkyl, hydroxyl, alkoxy, substituted alkoxy, halogen, and C(O)R⁸.
      A3. The method of embodiment A2, wherein R⁶ at each occurrence is selected from the group consisting of methyl, methoxy, perfluoromethyl, perfluoromethoxy, hydroxyl, Cl, F, and I.
      A4. The method of embodiment A1, wherein the compound has the structure of Formula II;

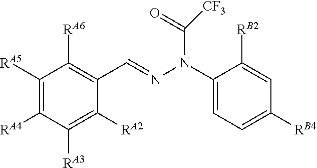

or a pharmaceutically acceptable salt, stereoisomer or tautomer thereof, wherein:
(i) R^{A2}, R^{A4}, R^{A5}, and R^{A6} is H, R^{A1} is methoxy, R^{B2} is methyl, and R^{B4} is methyl; or
(ii) R^{A2}, R^{A3}, R^{A5}, and R^{A6} is H, R^{A4} is methoxy, R^{B2} is methyl, and R^{B4} is methyl; or
(iii) R^{A2}, R^{A3}, R^{A4}, R^{A5}, and R^{A6} is H, R^{B2} is H, and R^{B4} is H; or
(iv) R^{A2}, R^{A3}, R^{A4}, R^{A5}, and R^{A6} is H, R^{B2} is methyl, and R^{B4} is methyl; or
(v) R^{A2}, R^{A4}, R^{A5}, and R^{A6} is H, R^{A3} is methoxy, R^{B2} is H, and R^{B4} is H; or
(vi) R^{A2}, R^{A3}, R^{A4}, R^{A5}, and R^{A6} is H, R^{B2} is H, and R^{B4} is methyl; or
(vii) R^{A2}, R^{A4}, R^{A5}, and R^{A6} is H, R^{A3} is methoxy, R^{B2} is H, and R^{B4} is methyl; or
(viii) R^{A2}, R^{A3}, R^{A4}, R^{A5}, and R^{A6} is H, R^{B2} is methyl, and R^{B4} is H; or
(ix) R^{A2}, R^{A4}, R^{A5}, and R^{A6} is H, R^{A3} is methoxy, R^{B2} is methyl, and R^{B4} is H; or
(x) R^{A2}, R^{A3}, R^{A5}, and R^{A6} is H, R^{A4} is COOH, R^{B2} is methyl, and R^{B4} is methyl; or
(xi) R^{A2}, R^{A4}, and R^{A5} is H, R^{A3} and R^{A6} is hydroxyl, R^{B2} is methyl, and R^{B4} is methyl; or
(xii) R^{A2}, R^{A4}, and R^{A6} is H, R^{A3} and R^{A5} is hydroxyl, R^{B2} is methyl, and R^{B4} is methyl; or
(xiii) R^{A2}, R^{A4}, and R^{A5} is H, R^{A3} is methoxy, R^{A6} is F, R^{B2} is H, and R^{B4} is Cl; or
(xiv) R^{A3} and R^{A5} is H, R^{A2} and R^{A6} is F, R^{A4} is hydroxyl, R^{A6} is F, R^{B2} is H, and R^{B4} is F; or
(xv) R^{A2}, R^{A4}, and R^{A6} is H, R^{A3} is hydroxyl, R^{A5} is F, R^{B2} is H, and R^{B4} is F; or
(xvi) R^{A2}, R^{A5}, and R^{A6} is H, R^{A3} and R^{A4} taken together are —O—CH₂—O—, R^{A5} is F, R^{B2} is H, and R^{B4} is F.
   A5. The method of embodiment A4, wherein R^{A2}, R^{A4}, R^{A5}, and R^{A6} is H, R^{A3} is methoxy, R^{B2} is methyl, and R^{B4} is methyl.
   B1. A method of preventing or treating PICCD in a subject comprising administering to the subject a therapeutically effective amount of a compound comprising a structure of Formula IV;

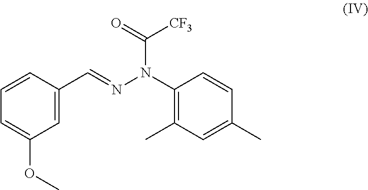 [SMILES: COc1cccc(/C=N/N(C(=O)C(F)(F)F)c2ccc(C)cc2C)c1]

or a pharmaceutically acceptable salt, stereoisomer or tautomer thereof.
C1. A method of preventing or treating PICCD in a subject comprising administering to the subject a therapeutically effective amount of a compound comprising a structure selected from any one of Formulas I to IV.
C1.1. The method of any one of embodiments A1 to C1, wherein the PICCD comprises cognitive and/or mental impairment.
C1.2. The method of any one of embodiments A1 to C1.1, wherein the PICCD is post-acute care syndrome.
C1.3. The method of any one of embodiments A1 to C1.2, wherein the PICCD comprises delirium, memory loss, a confusional state, reduced awareness, impaired executive function, impaired language, loss of attention and/or an impairment of visual-spatial abilities.
C1.4. The method of any one of embodiments A1 to C1.3, wherein the PICCD is acute, transient or temporary.
C1.5. The method of any one of embodiments A1 to C1.4, wherein the PICCD is chronic.
C2. The method of any one of embodiments A1 to C1.5, wherein the subject is human.
C2.1. The method of any one of embodiments A1 to C2, wherein the subject is elderly or at least 60 years of age.
C2.2. The method of any one of embodiments A1 to C2.1, wherein the subject is a pediatric patient or less than 13 years of age.
C2.3. The method of any one of embodiments A1 to C2.2, wherein the subject is female.
C2.4. The method of any one of embodiments A1 to C2.3, wherein the subject is at risk of being intubated, and/or at risk of being operably connected to a ventilator.
C2.5. The method of any one of embodiments A1 to C2.4, wherein the subject is intubated, and/or is operably connected to a ventilator.
C2.6. The method of any one of embodiments A1 to C2.5, wherein the subject was intubated, and/or was operably connected to a ventilator.
C3. The method of any one of embodiments C1 to C2.6, wherein the compound comprises a structure of Formula III;

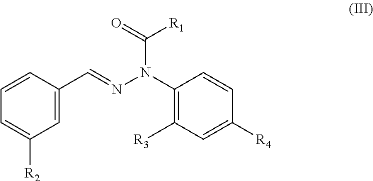

or a pharmaceutically acceptable salt, stereoisomer or tautomer thereof, wherein R₁ is methyl, fluoromethyl, difluoromethyl, trifluoromethyl, bromomethyl, dibromomethyl or tribromomethyl; R₂ is OCH₃, OCF₃ or OCBr₃; and R₃ and R₄ are independently selected from hydrogen, hydroxyl, a halogen (e.g., Cl, F or Br), methyl, a methoxy, or an amine.
C4. The method of embodiment C3, wherein the compound comprises the structure of Formula IV;

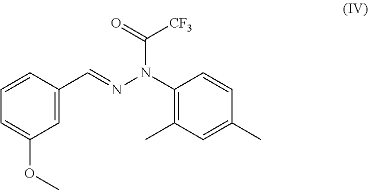 [SMILES: COc1cccc(/C=N/N(C(=O)C(F)(F)F)c2ccc(C)cc2C)c1]

or a pharmaceutically acceptable salt, stereoisomer or tautomer thereof.
C5. The method of any one of embodiments A1 to C4, wherein the PICCD results from, or is induced by a stay in an intensive care unit.
C6. The method of any one of embodiments A1 to C5, wherein the PICCD results from, or is induced by intubation of the subject.
C7. The method of any one of embodiments A1 to C6, wherein the subject displays stable cognitive function prior to intubation, prior to a stay in an ICU or prior to being operably connected to a ventilator.
C8. The method of any one of embodiments A1 to C7, wherein the subject was not diagnosed with a cognitive disorder or neurodegenerative disease prior to being intubated or prior to being operably connected to a ventilator.
C9. The method of any one of embodiments A1 to C7, wherein the subject is diagnosed with a cognitive disorder or neurodegenerative disease prior to being intubated or prior to being operably connected to a ventilator.
C10. The method of embodiment C8 or C9, wherein the cognitive disorder or neurodegenerative disease is selected from Alzheimer's disease, Parkinson's disease, Huntington's disease, amyotrophic lateral sclerosis, glaucoma, retinal degeneration, macular degeneration, age-related hearing loss, mild cognitive impairment, dementia, delirium, progressive supranuclear palsy, spinocerebellar ataxia, retinal neuropathy, peripheral neuropathy, diabetic neuropathy, background neuropathy, familial amyloid polyneuropathy, systemic senile amyloidosis, prion disease, scrapie, bovine spongiform encephalopathy, Creutzfeldt-Jakob disease, Gerstmann-Straussler-Scheinker syndrome and amyloidosis.
C11. The method of any one of embodiments A1 to C10, wherein the subject was not previously diagnosed with, and/or does not have cancer, diabetes, arthritis, insulinoma stroke, ischemia (e.g., heart ischemia) or cardiovascular disease.
C12. The method of any one of embodiments A1 to C10, wherein the subject was previously diagnosed with, has or is suspected of having cancer, diabetes, hyperglycemia, hypoglycemia, fluctuation of serum glucose, in-house acute stress syndrome, delirium, arthritis, pancreatitis and/or insulinoma.
C13. The method of any one of embodiments A1 to C12, wherein the subject has or is risk of acute respiratory syndrome.
C14. The method of any one of embodiments A1 to C13, wherein the subject has or has been diagnosed with asthma.
C15. The method of any one of embodiments A1 to C14, wherein the subject has or has been diagnosed with pneumonia.
C16. The method of any one of embodiments A1 to C15, wherein the subject is infected with a pathogen.
C17. The method of any one of embodiments A1 to Cl6, wherein the pathogen is a virus, fungus or bacteria.
C18. The method of embodiment C17, wherein the virus is a coronavirus.
C19. The method of embodiment C18, wherein the coronavirus is COVID19.
C20. The method of any one of embodiments A1 to C19, wherein the subject was previously diagnosed with, or is prone to, depression.
C21. The method of any one of embodiments A1 or C20, wherein the subject was not previously administered (e.g., prior to a stay in an ICU or prior to intubation) a compound selected from any one of Formula I, Formula II, Formula III and Formula IV.
C22. The method of any one of embodiments A1 or C21, wherein the compound is administered to the subject prior to, during, and/or after the subject is intubated, or operably connected to a ventilator.
C23. The method of any one of embodiments A1 or C22, wherein the compound is administered to the subject prior to, during, and/or after the subject is in, or admitted to an intensive care unit.
C24. The method of any one of embodiments A1 to C23, wherein the compound is administered at least 24 hours, at least 12 hours or at least 4 hours prior to the intubation, prior to the operable connection to a ventilator, or prior to a stay or admittance to an ICU.
C25. The method of any one of embodiments A1 to C24, wherein the compound is administered at an interval of once or twice per day.
C26. The method of any one of embodiments A1 to C25, wherein the compound is administered at a dose of 0.5 mg/kg to 100 mg/kg, or 10 mg/kg to 50 mg/kg.
C27. The method of any one of embodiments A1 to C26, wherein the compound is administered orally or intravenously.
D1. A compound comprising a structure selected from any one of Formula I, Formula II, Formula III and Formula IV for use in conducting a method of any one of embodiments A1 to C27.
D2. A pharmaceutical composition comprising a compound comprising a structure selected from any one of Formula I, Formula II, Formula III and Formula IV for use in conducting a method of any one of embodiments A1 to C27.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in some embodiments or aspects of the methods disclosed herein, some materials and/or method steps are excluded. Thus, even though the invention is generally not expressed herein in terms of what the invention does not include aspects that are not expressly excluded in the invention are nevertheless disclosed herein.

Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

1-59. (canceled)

60. A method of preventing, reducing the severity of, delaying the onset of, or treating Post-Intensive Care Cognitive Dysfunction (PICCD) in a subject or in a subject at risk of developing PICCD, comprising administering to the subject, or the subject at risk, a therapeutically effective amount of a compound comprising the structure of Formula I, 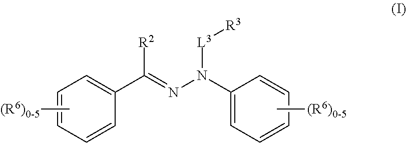
or a pharmaceutically acceptable salt, stereoisomer or tautomer thereof,
wherein:
R² is hydrogen or methyl;
R³ is trifluoromethyl or other fluoro substituted alkyl;
L³ is a carbonyl;
R⁶ at each occurrence is independently selected from the group consisting of alkyl, substituted alkyl, cycloalkyl, substituted cycloalkyl, hydroxyl, alkoxy, substituted alkoxy, aryloxy, substituted aryloxy, mercapto, alkylthio, arylthio, carbonyl, aryl, substituted aryl, substituted heterocyclic, halogen, cyano, cyanoalkyl, nitro, amino, amidino, carbamate, S(O)ₙR⁷ and C(O)R⁸ or two R⁶ at adjacent positions combine to form an optionally substituted heteroaryl or heteroalkyl ring fused with the adjoining phenyl moiety;
R⁷ is H, R⁹, NH₂, HNR⁹ or NR⁹R¹⁰;
R⁸ is OH, OR⁹, NH₂, NHR⁹ or NR⁹R¹⁰;
R⁹ and R¹⁰ at each occurrence are independently optionally substituted alkyl; and
n=1 or 2.

61. The method of claim 60, wherein the compound has the structure of Formula II; 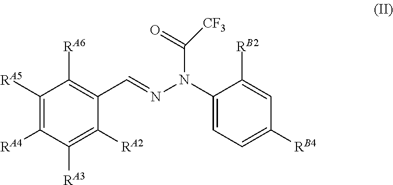
or a pharmaceutically acceptable salt, stereoisomer or tautomer thereof,
wherein:
(i) R^{A2}, R^{A4}, R^{A5}, and R^{A6} is H, R^{A3} is methoxy, R^{B2} is methyl, and R^{B4} is methyl; or
(ii) R^{A2}, R^{A3}, R^{A5}, and R^{A6} is H, R^{A4} is methoxy, R^{B2} is methyl, and R^{B4} is methyl; or
(iii) R^{A2}, R^{A3}, R^{A4}, R^{A5}, and R^{A6} is H, R^{B2} is H, and R^{B4} is H; or
(iv) R^{A2}, R^{A3}, R^{A4}, R^{A5}, and R^{A6} is H, R^{B2} is methyl, and R^{B4} is methyl; or
(v) R^{A2}, R^{A4}, R^{A5}, and R^{A6} is H, R^{A3} is methoxy, R^{B2} is H, and R^{B4} is H; or
(vi) R^{A2}, R^{A3}, R^{A4}, R^{A5}, and R^{A6} is H, R^{B2} is H, and R^{B4} is methyl; or
(vii) R^{A2}, R^{A4}, R^{A5}, and R^{A6} is H, R^{A3} is methoxy, R^{B2} is H, and R^{B4} is methyl; or
(viii) R^{A2}, R^{A3}, R^{A4}, R^{A5}, and R^{A6} is H, R^{B2} is methyl, and R^{B4} is H; or
(ix) R^{A2}, R^{A4}, R^{A5}, and R^{A6} is H, R^{A3} is methoxy, R^{B2} is methyl, and R^{B4} is H; or
(x) R^{A2}, R^{A3}, R^{A5}, and R^{A6} is H, R^{A4} is COOH, R^{B2} is methyl, and R^{B4} is methyl; or
(xi) R^{A2}, R^{A4}, and R^{A5} is H, R^{A3} and R^{A6} is hydroxyl, R^{B2} is methyl, and R^{B4} is methyl; or
(xii) R^{A2}, R^{A4}, and R^{A6} is H, R^{A3} and R^{A5} is hydroxyl, R^{B2} is methyl, and R^{B4} is methyl; or
(xiii) R^{A2}, R^{A4}, and R^{A5} is H, R^{A3} is methoxy, R^{A6} is F, R^{B2} is H, and R^{B4} is Cl; or
(xiv) R^{A3} and R^{A5} is H, R^{A2} and R^{A6} is F, R^{A4} is hydroxyl, R^{A6} is F, R^{B2} is H, and R^{B4} is F; or
(xv) R^{A2}, R^{A4}, and R^{A6} is H, R^{A3} is hydroxyl, R^{vs} is F, R^{B2} is H, and R^{B4} is F; or
(xvi) R^{A2}, R^{A5}, and R^{A6} is H, R^{A3} and R^{A4} taken together are —O—CH₂—O—, R^{A5} is F, R^{B2} is H, and R^{B4} is F.

62. The method of claim 60, wherein the compound has the structure of Formula III; 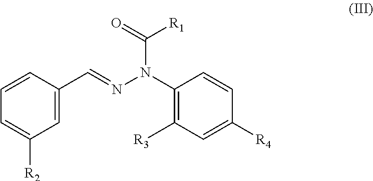
or a pharmaceutically acceptable salt, stereoisomer or tautomer thereof, wherein
R₁ is methyl, fluoromethyl, difluoromethyl, trifluoromethyl, bromomethyl, dibromomethyl or tribromomethyl;
R² is methyl, methoxy, hydroxyl, halogen, CF₃, OCH₃, OCF₃ or OCBr₃;
L³ is a carbonyl;
R₃ and R₄ are independently selected from the group consisting of hydrogen, hydroxyl, halogen, methyl, methoxy, and an amine.

63. The method of claim 60, wherein the compound has the structure of Formula IV; 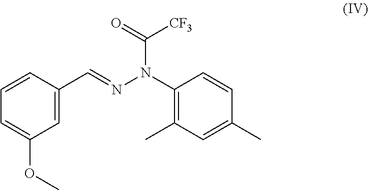 [SMILES: COc1cccc(/C=N/N(C(=O)C(F)(F)F)c2ccc(C)cc2C)c1]
or a pharmaceutically acceptable salt, stereoisomer or tautomer thereof.

64. The method of claim 60, wherein the subject, or subject at risk, is at risk of being, is, or was (a) admitted to an intensive care unit, (b) intubated, or (c) operably connected to a ventilator.

65. The method of claim 60, wherein the subject, or subject at risk, was previously diagnosed with, has, or is suspected of having hyperglycemia, hypoglycemia, fluctuation of serum glucose, pancreatitis, in-house acute stress syndrome, depression, anxiety and/or delirium.

66. The method of claim 60, wherein the subject, or subject at risk, has, is suspected of having, or is at risk of having acute respiratory syndrome, acute respiratory distress syndrome (ARDS), severe acute respiratory syndrome (SARS), asthma, pneumonia, or an infection.

67. The method of claim 60, wherein the subject is infected with a pathogen selected from viruses, fungi and bacteria.

68. The method of claim 67, wherein the virus is SARS-associated coronavirus or SARS-associated coronavirus-2.

69. The method of claim 60, wherein the PICCD comprises a presentation of, or worsening of: delirium, memory loss, a confusional state, reduced awareness, impaired executive function, impaired speech, impaired language, impaired communication, loss of attention, depression, anxiety, post-traumatic stress disorder and/or an impairment of visual-spatial abilities.

70. The method of claim 60, wherein the subject, or subject at risk, is at least 60 years of age.

71. A method of treating, preventing, inhibiting, reducing the severity of, or delaying the onset of a cognitive impairment or a cognitive disorder resulting from, or caused by respiratory distress in a subject, the method comprising administering a therapeutically effective amount of a compound to a subject who is at risk of having respiratory distress, who has or is experiencing respiratory distress, or who has previously experienced respiratory distress; wherein the compound has the structure of Formula I 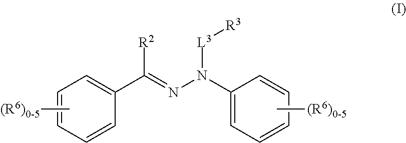
or a pharmaceutically acceptable salt, stereoisomer or tautomer thereof,
wherein:
R² is selected from the group consisting of H and methyl;
R³ is trifluoromethyl or other fluoro substituted alkyl;
L³ is a carbonyl;
R⁶ at each occurrence is independently selected from the group consisting of alkyl, substituted alkyl, cycloalkyl, substituted cycloalkyl, hydroxyl, alkoxy, substituted alkoxy, aryloxy, substituted aryloxy, mercapto, alkylthio, arylthio, carbonyl, aryl, substituted aryl, substituted heterocyclic, halogen, cyano, cyanoalkyl, nitro, amino, amidino, carbamate, S(O)ₙR⁷ and C(O)R⁸ or two R⁶ at adjacent positions combine to form an optionally substituted heteroaryl or heteroalkyl ring fused with the adjoining phenyl moiety;
R⁷ is H, R⁹, NH₂, HNR⁹ or NR⁹R¹⁰;
R⁸ is OH, OR⁹, NH₂, NHR⁹ or NR⁹R¹⁰;
R⁹ and R¹⁰ at each occurrence are independently optionally substituted alkyl; and
n=1 or 2.

72. A method of treating a subject who is at risk of having respiratory distress, who has or is experiencing respiratory distress, or who has previously experienced respiratory distress comprising administering a therapeutically effective amount of a compound to the subject, wherein a cognitive impairment or a cognitive disorder resulting from, or caused by the respiratory distress is prevented, ameliorated, inhibited, reduced in severity, or delayed; wherein the compound has the structure of Formula I 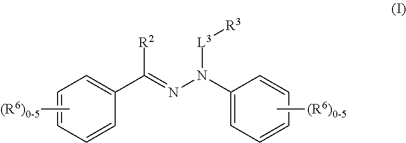
or a pharmaceutically acceptable salt, stereoisomer or tautomer thereof,
wherein:
R² is selected from the group consisting of H and methyl;
R³ is trifluoromethyl or other fluoro substituted alkyl;
L³ is a carbonyl;
R⁶ at each occurrence is independently selected from the group consisting of alkyl, substituted alkyl, cycloalkyl, substituted cycloalkyl, hydroxyl, alkoxy, substituted alkoxy, aryloxy, substituted aryloxy, mercapto, alkylthio, arylthio, carbonyl, aryl, substituted aryl, substituted heterocyclic, halogen, cyano, cyanoalkyl, nitro, amino, amidino, carbamate, S(O)ₙR⁷ and C(O)R⁸ or two R⁶ at adjacent positions combine to form an optionally substituted heteroaryl or heteroalkyl ring fused with the adjoining phenyl moiety;
R⁷ is H, R⁹, NH₂, HNR⁹ or NR⁹R¹⁰;
R⁸ is OH, OR⁹, NH₂, NHR⁹ or NR⁹R¹⁰;
R⁹ and R¹⁰ at each occurrence are independently optionally substituted alkyl; and
n=1 or 2.

73. The method of claim 71, wherein the respiratory distress is selected from acute respiratory distress, acute respiratory distress syndrome (ARDS), and severe acute respiratory syndrome (SARS).

74. The method of claim 71, wherein the respiratory distress is associated with hypoxia.

75. The method of claim 71, wherein the cognitive impairment comprises delirium, memory loss, a confusional state, reduced awareness, impaired executive function, impaired language, loss of attention and/or an impairment of visual-spatial abilities.

76. The method of claim 71, wherein the subject is at least 60 years of age.

77. The method of claim 71, wherein the subject was previously diagnosed with, has, or is suspected of having cancer, diabetes, hyperglycemia, hypoglycemia, fluctuation of serum glucose, in-house acute stress syndrome, delirium, arthritis, pancreatitis and/or insulinoma.

78. The method of claim 71, wherein the subject has, or has been diagnosed with, asthma.

79. The method of claim 71, wherein the subject has, or has been diagnosed with, pneumonia.

80. The method of claim 71, wherein the subject is infected with a pathogen selected from viruses, fungi and bacteria.

81. The method of claim 80, wherein the virus is SARS-associated coronavirus or SARS-associated coronavirus-2.

82. The method of claim 73, wherein the respiratory distress is acute respiratory distress syndrome (ARDS).

83. The method of claim 82, wherein the ARDS is associated with or caused by sepsis, pneumonia, a lung infection, pancreatitis, physical trauma, aspiration, smoke inhalation, toxic substance inhalation, idiopathic pulmonary fibrosis, blood transfusion, massive blood transfusion, burns, near-drowning, reactions to medications, drug overdose, shock, lung surgery, cardiopulmonary bypass surgery, disseminated intravascular coagulation, or tick-born relapsing fever.

84. The method of claim 71, wherein the compound is N-(2,4-dimethylphenyl)-2,2,2-trifluoro-N′-[(E)-(3-methoxyphenyl)methylene]acetohydrazide (also J147) or a pharmaceutically acceptable salt, stereoisomer or tautomer thereof.
